(19) United States
(12) Patent Application Publication
(10) Pub. No.: US 2010/0216823 A1
(43) Pub. Date: Aug. 26, 2010

(54) Spirocyclic Derivatives

(75) Inventors: David James Rawson, Kent (GB); Nigel Alan Swain, Kent (GB); Lesa Watson, Kent (GB)

Correspondence Address: PFIZER INC.;PATENT DEPARTMENT, Bld 114 M/S 9114, EASTERN POINT ROAD, GROTON, CT, 06340, US

(73) Assignee: Pfizer Inc.

(21) Appl. No.: 12/600,714

(22) PCT Filed: May 16, 2008
(86) PCT No.: PCT/IB2008/001295

§ 371 Date: Apr. 26, 2010

Related U.S. Application Data
(60) Provisional application No. 60/939,925, filed on May 24, 2007.

(51) Int. Cl. A61K 31/527; C07D 239/80; C07D 403/10; C07D 413/10; A61P 29/00; A61P 35/00; A61P 37/06; A61P 25/28; A61P 11/00; A61P 11/06; A61P 19/00

(52) U.S. Cl. 514/266.3; 544/231

(57) ABSTRACT

The invention provides compounds of formula (I):

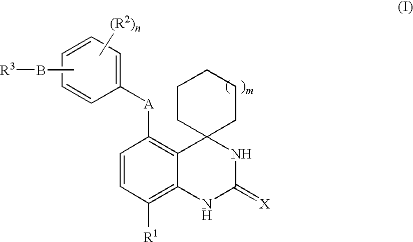

wherein m, n, X, R¹, A, B, R² and R³ have the meanings given in the specification, and pharmaceutically acceptable salts, solvates, polymorphs and prodrugs thereof. The compounds are PDE7 inhibitors and have a number of therapeutic applications, particularly in the treatment of pain, especially neuropathic pain.

### FIELD OF THE INVENTION

This invention relates to spirocyclic derivatives, and to processes for the preparation of, intermediates used in the preparation of, compositions containing and the uses of, such derivatives.

The spirocyclic derivatives of the present invention are PDE7 inhibitors and have a number of therapeutic applications, particularly in the treatment of pain, especially neuropathic pain.

### BACKGROUND TO THE INVENTION

Phosphodiesterases (PDEs) are a family of enzymes which affect various cellular signalling processes by the process of hydrolyzing the second messenger molecules cAMP and cGMP to the corresponding inactive 5′-monophosphate nucleotides and thereby regulating their physiological level. The secondary messengers cAMP and cGMP are responsible for the regulation of numerous intracellular processes. There are at least 11 families of PDE's, some (PDE3, 4, 7, 8) being specific for cAMP, and others for cGMP (PDE5, 6, and 9).

PDE7 is one member of the PDE family and comprises 2 subclass members PDE7 A and B. The mRNA of PDE7 is expressed in various tissues and cell types known to be important in the pathogenesis of several diseases such as T-cell related disorders. In particular PDE7A and its splice variants are upregulated in activated T-cells, (L. Li, C. Yee and J. A. Beavo, Science (1999), 283, 848-851), and in B-lymphocytes. (R. Lee, S. Wolda, E. Moon, J. Esselstyn, C. Hertel and A. Lerner, Cell. Signal (2002), 14, 277-284), autoimmune disease (L. Li et al, above), and airway disease (S. J. Smith et al, Am. J. Physiol. Lung. Cell. Mol. Physiol. (2003), 284, L279-L289). Consequently it is expected that selective inhibitors of PDE7 will have broad application as both immunosuppressants and treatment for respiratory conditions, for example chronic obstructive pulmonary disease and asthma (N. A. Glavas, C. Ostenson, J. B. Schaefer, V. Vasta and J. A. Beavo. PNAS (2001), 98, 6319-6324).

Studies in rat have shown that PDE7A mRNA is found to be widely distributed in rat brain in both neuronal and non-neuronal cell populations. The highest levels are observed in the olfactory bulb, olfactory tubercle, hippocampus, cerebellum, medial habenula nucleus, pineal gland, area postrema, and choroid plexus. PDE7A mRNA is also widely detected in other non brain tissue. These results are consistent with PDE7A being involved in the regulation of cAMP signaling in many brain functions and suggests that PDE7A could have an effect on memory, depression, and emesis (X. Miró, S. Pérez-Torres, J. M. Palacios, P. Puigdoménech, G. Mengod, Synapse (2001), 40, 201-214). A link to Alzheimer's disease is also suggested (S. Pérez Torres et al, Experimental Neurology, (2003) 182, 322-334). Additionally PDE7 has also been implicated in both fertility disorders (WO 01/83772) and leukaemia (R. Lee et al., Cell Signalling (2002) 14, 277-284).

PDE7A has been isolated from yeast (T. Michaeli et al, J. Biol. Chem. (1993) 268, 12925-12932), human (P. Han, Z. Xiaoyan and, M. Tamar, J. Biol. Chem. (1997) 272, 16152-16157), mouse (T. Bloom and J. A. Beavo, Proc. Natl. Acad. Sci. USA (1996), 93, 14188-14192) and upregulation of PDE7A levels is seen in human T lymphocytes (M. Ichimura and H. Kase. Biochem. Biophys. Res. Commun. (1993), 193, 985-990).

PDE7B, the second member of the PDE7 family, shares 70% amino acid homology with PDE7A in the C-terminal catalytic domain (N-terminal domain is the regulatory domain containing the phosphorylation site which is conserved across the PDE family). PDE7B is cAMP specific and has been cloned from mouse (accession number—AJ251858) and human (accession number—AJ251860) sources (C. Gardner, N. Robas, D. Cawkill and M. Fidock, Biochem. Biophys. Res. Commun. (2000), 272, 186-192). It has been shown to be expressed in a wide variety of tissues: the caudate nucleus, putamen and occipital lobe of the brain and peripherally in the heart, ovary and pituitary gland, kidney and liver small intestine and thymus, additionally in skeletal muscle, colon, bladder, uterus, prostate, stomach adrenal gland and thyroid gland. PDE7B has also been shown to discriminate among several general PDE inhibitors (J. M. Hetman, S. H. Soderling, N. A. Glavas and J. A. Beavo, PNAS (2000), 97, 472-476). However, many standard PDE inhibitors, such as zaprinast, rolipram and milrinone, do not specifically inhibit PDE7B.

Inhibitors of PDE7 are known as is their use in the treatment of various PDE7 related diseases. For example, WO 02/074754 describes compounds of formulae:

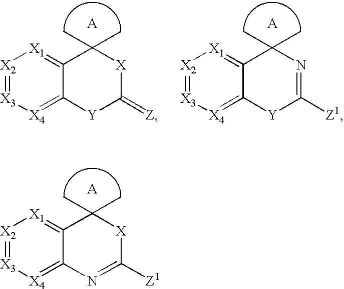

and their use in the treatment of PDE7-related disorders, such as T-cell-related diseases, autoimmune diseases, osteoarthritis, multiple sclerosis, osteoporosis, chronic obstructive pulmonary disease, asthma, cancer, acquired immune deficiency syndrome, allergy or inflammatory bowel disease.

WO 2004/026818 describes compounds of formula:

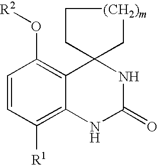

and their use in the treatment of PDE7-related disorders.

WO 2006/092691 describes the use of PDE7 inhibitors in the treatment of neuropathic pain.

International Patent Application No. PCT/IB2006/003388, published as WO 2007/063391, describes compounds of formula:

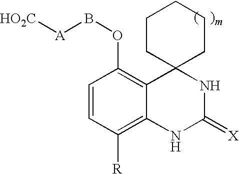

and their use in the treatment of PDE7-related disorders, including pain.

PDE1 isoforms are expressed in the brain, in myocardial cells and in vascular smooth muscle cells. The three subtypes of PDE1, namely PDE1A, PDE1B, and PDE1C are all Ca²⁺-calmodulin activated, and are known to be activated by vasoconstricting agents such as noradrenaline and angiotensin (Rybalkin et al., Cyclic GMP Phosphodiesterases and Regulation of Smooth Muscle Function. Circulation Research 2003; 93:280-). Inhibition of any of the subunits, including PDE1C, is likely to prevent the increase in vascular tone induced by endogenous vasoconstrictor agents and, in a tonically active system, may lead to vasodilatation, flushing, and tachycardia. (Giembycz Current Opinion in Pharmacology, 5(3), 2005, 238-244) Chronic vasodilatation via non-selective PDE inhibition is known to induce lesions of the cardiovascular system, thus increased selectivity over PDE1 isoforms is likely to lead to a decreased probability of cardiovascular toxicity.

We have surprisingly found that a class of compounds falling within the general disclosure of WO 02/074754, but not specifically disclosed or exemplified therein, exhibit unexpectedly superior selectivity for inhibition of the PDE7A enzyme over the PDE1C enzyme, when compared with the closest compounds exemplified in WO 02/074754. This increased selectivity is likely to lead to the compounds exhibiting a decreased probability of cardiovascular toxicity in patients than the closest prior art compounds.

### SUMMARY OF THE INVENTION

The invention provides a compound of formula (I):

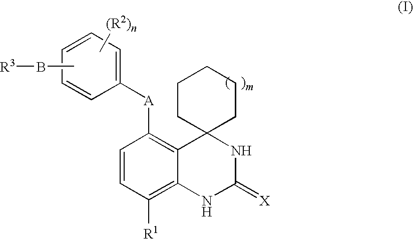

wherein:
m is 0, 1 or 2;
n is 0, 1, 2 or 3;

### X is O, S or N—CN;

R¹ is halogen or CN;
A is a single bond, CH₂, O or S;
B is a single bond, CH₂ or OCH₂;
each R² is independently halogen, (C₁₋₆)alkyl (optionally substituted by 1 to 3 fluorine atoms), OH, (C₁₋₆)alkoxy, (C₁₋₆)alkylthio or CN;
R³ is selected from the following groups (i) to (x):

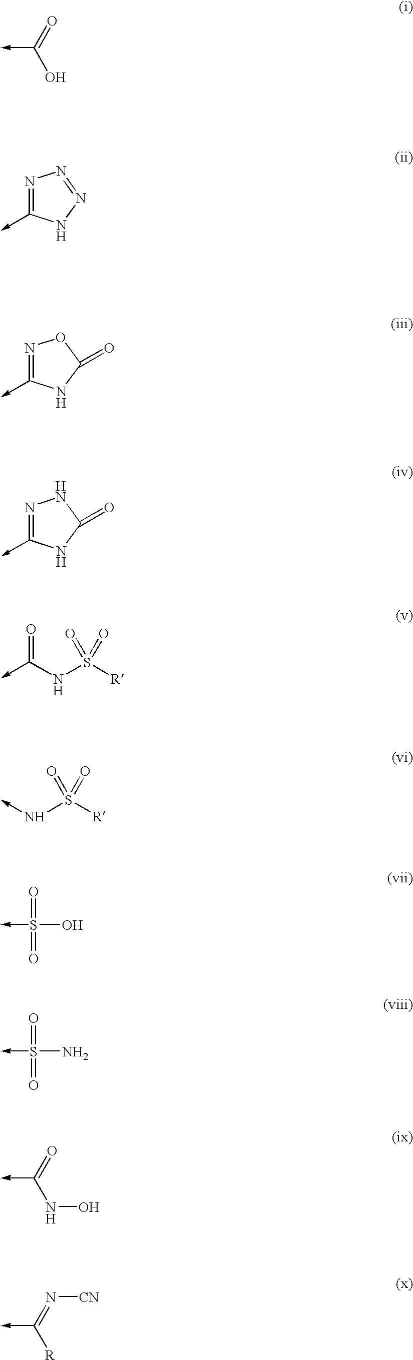

R is H or (C₁₋₆)alkyl (optionally substituted by 1 to 3 fluorine atoms);
R′ is (C₁₋₆)alkyl (optionally substituted by 1 to 3 fluorine atoms);
or a pharmaceutically acceptable salt, solvate, polymorph or prodrug thereof.

### DETAILED DESCRIPTION OF PREFERRED EMBODIMENTS

In the context of the present invention, the term “alkyl” denotes a monovalent, straight or branched, saturated hydrocarbon chain containing 1 to 6 carbon atoms. Examples of alkyl groups include methyl, ethyl, n-propyl, isopropyl, n-butyl, isobutyl, sec-butyl, tert-butyl, n-pentyl, 2-methylbutyl, 3-methylbutyl, neopentyl, n-hexyl, 2-methylpentyl, 3-methylpentyl, 4-methylpentyl, 2-ethylbutyl and 2,2-dimethylbutyl. Preferred alkyl groups are (C₁₋₄)alkyl groups, particularly methyl and ethyl, especially methyl.

Where stated, alkyl groups may be substituted by 1 to 3 fluorine atoms. The substitution may be at any position on the alkyl chain. Preferably, such fluorinated alkyl groups have 1 to 4 carbon atoms, more preferably 1 or 2 carbon atoms. Mono-, di- and trifluoromethyl groups (especially trifluoromethyl), and mono-, di- and trifluoroethyl groups (especially 2,2,2-trifluoroethyl) are especially preferred.

The term “alkoxy” denotes “alkyl-O—”, wherein “alkyl” is as defined above, either in its broadest aspect or a preferred aspect. Preferred alkoxy groups are (C₁₋₄)alkoxy groups, particularly methoxy and ethoxy.

The term “alkylthio” denotes “alkyl-S—”, wherein “alkyl” is as defined above, either in its broadest aspect or a preferred aspect. Preferred alkylthio groups are (C₁₋₄)alkylthio groups, particularly methylthio and ethylthio.

The term “halogen” denotes fluoro, chloro, bromo or iodo. Preferred halogen groups are fluoro and chloro.

Preferably, m is 0 or 1, more preferably 1.

Preferably, n is 0 or 1, more preferably 0.

Preferably, X is O or N—CN, more preferably O.

Preferably, R¹ is F or Cl, more preferably Cl.

Preferably, A is a single bond or O, more preferably O.

When the group B is OCH₂, the oxygen atom is bonded to the benzene ring and the methylene group to the group R³.

Preferably, B is a single bond.

Preferably, R² is F or Cl, more preferably F.

Preferably, R³ is a group (i), (ii), (iii), (iv), (v) or (vi), more preferably a group (i) or (ii), and especially a group (ii).

In one embodiment, the group —B—R³ is present at the 2-position of the phenyl ring (the position of the group A being the 1-position). In other embodiments, the group —B—R³ is present at the 3-position. In further embodiments, the group —B—R³ is present at the 4-position.

Particularly preferred compounds of the invention include those in which each variable in Formula (I) is selected from the suitable and/or preferred groups for each variable. Even more preferred compounds of the invention include those where each variable in Formula (I) is selected from the more preferred or most preferred groups for each variable.

The following compounds are preferred:
5-[(8′-chloro-2′-oxo-2′,3′-dihydro-1′H-spiro[cyclohexane-1,4′-quinazolin]-5′-yl)]-2-fluorobenzoic acid;
3-(8′-chloro-2-oxo-2′,3′-dihydro-1′H-spiro[cyclohexane-1,4′-quinazolin]-5′-ylbenzoic acid;
5-[(8′-chloro-2′-oxo-2′,3′-dihydro-1′H-spiro[cyclohexane-1,4′-quinazolin]-4′-yl)]-2-fluorobenzoic acid;
8′-chloro-5′-[4-fluoro-3-(2H-tetrazol-5-yl)phenyl]-1′H-spiro[cyclohexane-1,4′-quinazolin]-2′(3′H)-one;
[3-(8′-chloro-2′-oxo-2′,3′-dihydro-1′H-spiro[cyclohexane-1,4′-quinazolin]-5′-yl)phenoxy]acetic acid;
2-{(8′-chloro-2′-oxo-2′,3′-dihydro-1′H-spiro[cyclohexane-1,4′-quinazolin]-5′-yl)oxy}-3-fluorobenzoic acid;
2-{(8′-chloro-2′-oxo-2′,3′-dihydro-1′H-spiro[cyclopentane-1,4′-quinazolin]-5′-oxy}-3-fluorobenzoic acid;
3-chloro-2-{(8′-chloro-2′-oxo-2′,3′-dihydro-1′H-spiro[cyclohexane-1,4′-quinazolin]-5′-yl)oxy}benzoic acid;
3-chloro-2-{(8′-fluoro-2′-oxo-2′,3′-dihydro-1′H-spiro[cyclohexane-1,4′-quinazolin]-5′-yl)oxy}benzoic acid;
8′-chloro-5′-[2-fluoro-6-(2H-tetrazol-5-yl)phenoxy]-1′H-spiro[cyclohexane-1,4′-quinazolin]-2′(3′H)-one;
8′-chloro-5′-[4-fluoro-2-(1H-tetrazol-5-yl)phenoxy]-1′H-spiro[cyclohexane-1,4′-quinazolin]-2′(3′H)-one;
8′-chloro-5′-[6-fluoro-2-(1H-tetrazol-5-yl)phenoxy]-1′H-spiro[cyclohexane-1,4′-quinazolin]-2′(3′H)-one;
8′-chloro-5′-[4-fluoro-2-(1H-tetrazol-5-yl)phenoxy]-1′H-spiro[cyclopentane-1,4′-quinazolin]-2′(3′H)-one;
8′-chloro-5′-[6-fluoro-2-(1H-tetrazol-5-yl)phenoxy]-1′H-spiro[cyclopentane-1,4′-quinazolin]-2′(3′H)-one;
8′-chloro-5′-[6-chloro-2-(1H-tetrazol-5-yl)phenoxy]-1′H-spiro[cyclopentane-1,4′-quinazolin]-2′(3′H)-one;
8′-chloro-5′-[2-(1H-tetrazol-5-yl)phenoxy]-1′H-spiro[cyclopentane-1,4′-quinazolin]-2′(3′H)-one;
8′-chloro-5′-[2-(1H-tetrazol-5-yl)phenoxy]-1′H-spiro[cyclohexane-1,4′-quinazolin]-2′(3′H)-one;
8′-chloro-5′-[2-fluoro-6-(5-oxo-4,5-dihydro-1,2,4-oxadiazol-3-yl)phenoxy]-1′H-spiro[cyclohexane-1,4′-quinazolin]-2′(3′H)-one;
8′-chloro-5′-[2-fluoro-6-(5-oxo-4,5-dihydro-1H-1,2,4-triazol-3-yl)phenoxy]-1′H-spiro[cyclohexane-1,4′-quinazolin]-2′(3′H)-one;
2-[(8′-chloro-2′-oxo-2′,3′-dihydro-1′H-spiro[cyclohexane-1,4′-quinazolin]-5′-yl)oxy]-3-fluoro-N-(methylsulfonyl)benzamide;
N-{2-[(8′-chloro-2′-oxo-2′,3′-dihydro-1′H-spiro[cyclohexane-1,4′-quinazolin]-5′-yl)oxy]-3-fluorophenyl}-1,1,1-trifluoromethanesulfonamide;
{2-[(8′-chloro-2′-oxo-2′,3′-dihydro-1′H-spiro[cyclohexane-1,4′-quinazolin]-5′-yl)oxy]-3-fluorophenyl}acetic acid;
{2-[(8′-chloro-2′-oxo-2′,3′-dihydro-1′H-spiro[cyclohexane-1,4′-quinazolin]-5′-yl)oxy]phenoxy}acetic acid;
{4-[(8′-chloro-2′-oxo-2′,3′-dihydro-1′H-spiro[cyclohexane-1,4′-quinazolin]-5′-yl)oxy]phenoxy}acetic acid;
methyl 2-[(8′-chloro-2′-oxo-2′,3′-dihydro-1′H-spiro[cyclohexane-1,4′-quinazolin]-5′-yl)oxy]-3-fluorobenzoate;
   and pharmaceutically acceptable salts, solvates and prodrugs thereof.

The following compounds are more preferred:
8′-chloro-5′-[2-fluoro-6-(2H-tetrazol-5-yl)phenoxy]-1′H-spiro[cyclohexane-1,4′-quinazolin]-2′(3′H)-one;
8′-chloro-5′-[4-fluoro-2-(1H-tetrazol-5-yl)phenoxy]-1′H-spiro[cyclohexane-1,4′-quinazolin]-2′(3′H)-one;
8′-chloro-5′-[6-fluoro-2-(1H-tetrazol-5-yl)phenoxy]-1′H-spiro[cyclohexane-1,4′-quinazolin]-2′(3′H)-one;
8′-chloro-5′-[4-fluoro-2-(1H-tetrazol-5-yl)phenoxy]-1′H-spiro[cyclopentane-1,4′-quinazolin]-2′(3′H)-one;
8′-chloro-5′-[6-fluoro-2-(1H-tetrazol-5-yl)phenoxy]-1′H-spiro[cyclopentane-1,4′-quinazolin]-2′(3′H)-one;
8′-chloro-5′-[6-chloro-2-(1H-tetrazol-5-yl)phenoxy]-1′H-spiro[cyclopentane-1,4′-quinazolin]-2′(3′H)-one;
8′-chloro-5′-[2-(1H-tetrazol-5-yl)phenoxy]-1′H-spiro[cyclopentane-1,4′-quinazolin]-2′(3′H)-one;
8′-chloro-5′-[2-(1H-tetrazol-5-yl)phenoxy]-1′H-spiro[cyclohexane-1,4′-quinazolin]-2′(3′H)-one;
   and pharmaceutically acceptable salts, solvates and prodrugs thereof.

The following compounds are most preferred:
8′-chloro-5′-[2-fluoro-6-(2H-tetrazol-5-yl)phenoxy]-1′H-spiro[cyclohexane-1,4′-quinazolin]-2′(3′H)-one;
8′-chloro-5′-[4-fluoro-2-(1H-tetrazol-5-yl)phenoxy]-1′H-spiro[cyclohexane-1,4′-quinazolin]-2′(3′H)-one;
8′-chloro-5′-[6-fluoro-2-(1H-tetrazol-5-yl)phenoxy]-1′H-spiro[cyclopentane-1,4′-quinazolin]-2′(3′H)-one;
8′-chloro-5′-[2-(1H-tetrazol-5-yl)phenoxy]-1′H-spiro[cyclohexane-1,4′-quinazolin]-2′(3′H)-one;
   and pharmaceutically acceptable salts, solvates and prodrugs thereof.

The invention further comprises a pharmaceutical composition comprising a compound of formula (I), either in its broadest aspect or a preferred aspect, or a pharmaceutically acceptable salt, solvate, polymorph or prodrug thereof, and a pharmaceutically acceptable carrier or diluent.

The invention further comprises a compound of formula (I), either in its broadest aspect or a preferred aspect, or a pharmaceutically acceptable salt, solvate, polymorph or prodrug thereof, for use as a medicament.

The invention further comprises use of a compound of formula (I), either in its broadest aspect or a preferred aspect, or a pharmaceutically acceptable salt, solvate, polymorph or prodrug thereof, in the manufacture of a medicament for the treatment of diseases or conditions for which therapy by a PDE7 inhibitor is relevant.

The invention further comprises a compound of formula (I), either in its broadest aspect or a preferred aspect, or a pharmaceutically acceptable salt, solvate, polymorph or prodrug thereof, for use in the treatment of a disease or condition for which therapy by a PDE7 inhibitor is relevant.

The invention further comprises a method of treating a disease or condition for which therapy by a PDE7 inhibitor is relevant, comprising administering an effective amount of a compound of formula (I), either in its broadest aspect or a preferred aspect, or a pharmaceutically acceptable salt, solvate, polymorph or prodrug thereof.

The compounds of formula (I), being PDE7 inhibitors, are potentially useful in the treatment of a range of disorders. The treatment of pain, particularly neuropathic pain, is a preferred use.

Physiological pain is an important protective mechanism designed to warn of danger from potentially injurious stimuli from the external environment. The system operates through a specific set of primary sensory neurones and is activated by noxious stimuli via peripheral transducing mechanisms (see Millan, Prog. Neurobiol., (1999), 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (eg painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a hightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, Science, (2000), 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, Lancet, (1999) 353, 1959-1964). The symptoms of neuropathic pain are difficult to treat, as they are often heterogeneous even between patients with the same disease (Woolf & Decosterd, Pain Supp., (1999), 6, S141-S147; Woolf and Mannion, above). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, Ann Pharmacother., (2002), 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple aetiologies and thus can be classified in more than one area, e.g. back pain and cancer pain have both nociceptive and neuropathic components.

Other types of pain include:
pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache, tension-type headache mixed headache and headache associated with vascular disorders; and
orofacial pain, including dental pain, otic pain, burning mouth syndrome and temporomandibular myofascial pain.

The compounds of formula (I) of the present invention are also useful in the treatment of conditions other than pain. In particular, the compounds of formula (I) of the present invention are useful in the treatment of T-cell-related diseases, autoimmune diseases, multiple sclerosis, osteoporosis, chronic obstructive pulmonary disease, asthma, cancer, acquired immune deficiency syndrome (AIDS), allergy and inflammatory bowel disease.

The invention further comprises use of a compound of formula (I), either in its broadest aspect or a preferred aspect, or a pharmaceutically acceptable salt, solvate or prodrug thereof, in the manufacture of a medicament for the treatment of a condition or disorder selected from pain (especially neuropathic pain), T-cell-related diseases, autoimmune diseases, multiple sclerosis, osteoporosis, chronic obstructive pulmonary disease, asthma, cancer, acquired immune deficiency syndrome (AIDS), allergy and inflammatory bowel disease.

The invention also comprises a compound of formula (I), either in its broadest aspect or a preferred aspect, or a pharmaceutically acceptable salt, solvate or prodrug thereof, for use in the treatment of a condition or disorder selected from pain (especially neuropathic pain), T-cell-related diseases, autoimmune diseases, multiple sclerosis, osteoporosis, chronic obstructive pulmonary disease, asthma, cancer, acquired immune deficiency syndrome (AIDS), allergy and inflammatory bowel disease.

The invention additionally comprises a method of treating a disease or condition selected from pain (especially neuropathic pain), T-cell-related diseases, autoimmune diseases, multiple sclerosis, osteoporosis, chronic obstructive pulmonary disease, asthma, cancer, acquired immune deficiency syndrome (AIDS), allergy or inflammatory bowel disease, comprising administering an effective amount of a compound of formula (I), either in its broadest aspect or a preferred aspect, or a pharmaceutically acceptable salt, solvate or prodrug thereof.

Pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of formula (I) may be prepared by one or more of three methods:

(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water. The present invention embraces both the unsolvated and all solvated forms.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Hereinafter all references to compounds of formula (I) include references to salts and solvates thereof and to solvates of salts thereof.

The compounds of the invention include compounds of formula (I) as hereinbefore defined, including all polymorphs and crystal habits thereof, prodrugs and isomers thereof (including optical, geometric and tautomeric isomers) as hereinafter defined and isotopically-labelled compounds of formula (I).

As indicated, so-called ‘prodrugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as “prodrugs”. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

The compounds of formula (I) of the present invention contain a carboxylic acid functionality (—COOH). Therefore, suitable prodrugs comprise esters thereof, wherein the hydrogen of the carboxylic acid functionality of the compound of formula (I) is replaced by an ester residue. The term “ester residue” means an ester group which can be cleaved in vivo by a biological method such as hydrolysis and forms a compound of formula (I) having the free carboxylic acid group or a salt thereof.

Whether a compound is such a prodrug or not can, for example, be determined by administering it by intravenous injection to an experimental animal, such as a rat or mouse, and then studying the body fluids of the animal to determine whether or not the compound of formula (I) or a pharmaceutically acceptable salt thereof can be detected.

Preferred examples of the ester residue include:

C₁₋₂₀ alkyl groups, which may be straight or branched chain alkyl groups such as methyl, ethyl, n-propyl, isopropyl, n-butyl, isobutyl, sec-butyl, tert-butyl, n-pentyl, isopentyl, neopentyl, hexyl, heptyl, octyl, nonyl, decyl, undecyl, dodecyl, tridecyl, tetradecyl, pentadecyl, hexadecyl, heptadecyl, octadecyl, nonadecyl and icosanyl, especially C₁₋₁₂ alkyl groups, preferably C₁₋₈ alkyl groups, more preferably C₁₋₆alkyl groups, and most preferably C₁₋₄ alkyl groups such as those defined and exemplified above;

C₁₋₁₀ haloalkyl groups (defined as an alkyl group substituted by one or more halogen atoms, preferably fluorine or chlorine atoms, more preferably fluorine atoms), preferably C₁₋₈ haloalkyl groups, more preferably C₁₋₆ haloalkyl groups, and most preferably C₁₋₄ haloalkyl groups such as mono-, di- or trifluoromethyl, mono-, di- or trichloromethyl, bromomethyl, 2-fluoroethyl, 2,2-difluoroethyl, 2,2,2-trifluoroethyl, 2-chloroethyl, 2,2-dichloroethyl, 2,2,2-trichloroethyl, perfluoroethyl, perfluoropropyl and perfluorobutyl;

C₁₋₁₀ hydroxyalkyl groups (defined as an alkyl group substituted by a hydroxy (—OH) group), preferably C₁₋₈ hydroxyalkyl groups, more preferably C₁₋₆ hydroxyalkyl groups, and most preferably C₁₋₄ hydroxyalkyl groups such as hydroxymethyl, 1- or 2-hydroxyethyl, 1-, 2- or 3-hydroxypropyl, and 1-, 2-, 3- or 4-hydroxybutyl;

(C₁₋₁₀ alkoxy)C₁₋₁₀ alkyl groups (defined as an alkyl group substituted by an alkoxy group), preferably (C₁₋₆ alkoxy)C₁₋₆ alkyl groups, more preferably (C₁₋₄ alkoxy)C₁₋₄ alkyl groups, and most preferably (C₁₋₄ alkoxy)methyl groups, such as the methoxymethyl, 1,1-dimethyl-1-methoxymethyl, ethoxymethyl, propoxymethyl, isopropoxymethyl, butoxymethyl and t-butoxymethyl groups;

C₁₋₆ alkoxylated (C₁₋₆ alkoxy)methyl groups, such as the 2-methoxyethoxymethyl group;

halo(C₁₋₆ alkoxy)methyl groups, such as the 2,2,2-trichloroethoxymethyl and bis(2-chloroethoxy)methyl groups;

C₃₋₈ cycloalkyl groups, such as cyclopropyl, cyclobutyl, cyclopentyl, cyclohexyl, cycloheptyl and cyclooctyl groups;

aralkyl groups, for example: C₁₋₆ alkyl groups substituted by from 1 to 3 C₆₋₁₄ aryl groups (wherein the aryl part is selected from phenyl, naphthyl, anthryl and phenanthryl), such as the benzyl, α-naphthylmethyl, β-naphthylmethyl, diphenylmethyl, triphenylmethyl, α-naphthyldiphenylmethyl and 9-anthrylmethyl groups; and C₁₋₆ alkyl groups substituted by from 1 to 3 substituted C₆₋₁₄ aryl groups, where one or more of the aryl groups is substituted by one or more (preferably 1 to 3, and more preferably only 1) C₁₋₆ alkyl, C₁₋₆ alkoxy, nitro, halogen or cyano substituents, such as the 4-methylbenzyl, 2,4,6-trimethylbenzyl, 3,4,5-trimethylbenzyl, 4-methoxybenzyl, 4-methoxyphenyldiphenylmethyl, 2-nitrobenzyl, 4-nitrobenzyl, 4-chlorobenzyl, 4-bromobenzyl and 4-cyanobenzyl groups; especially the benzyl group;

tetrahydropyranyl or tetrahydrothiopyranyl groups, wherein the tetrahydropyranyl or tetrahydrothiopyranyl group may be optionally substituted with a substituent selected from halo and C₁₋₆ alkoxy, such as: tetrahydropyran-2-yl, 3-bromotetrahydropyran-2-yl, 4-methoxy-tetrahydropyran-4-yl, tetrahydrothiopyran-2-yl, and 4-methoxy-tetrahydrothiopyran-4-yl groups;

tetrahydrofuranyl or tetrahydrothiofuranyl groups, wherein the tetrahydrofuranyl or tetrahydrothiofuranyl group may be optionally substituted with a substituent selected from halo and C₁₋₆ alkoxy, such as: tetrahydrofuran-2-yl and tetrahydrothiofuran-2-yl groups;

C₂₋₁₀ alkenyl groups, such as the vinyl, propenyl, butenyl, pentenyl, hexenyl, heptenyl, octenyl, nonenyl and decenyl groups; and

C₂₋₁₀ alkynyl groups, such as ethynyl, propynyl, butynyl, pentynyl, hexynyl, heptynyl, octynyl, nonynyl and decynyl groups.

Further examples of replacement groups in accordance with the foregoing examples and examples of other prodrug types may be found in the aforementioned references.

Moreover, certain compounds of formula (I) may themselves act as prodrugs of other compounds of formula (I).

Compounds of formula (I) containing one or more asymmetric carbon atoms can exist as two or more stereoisomers. Where structural isomers are interconvertible via a low energy barrier, tautomeric isomerism (‘tautomerism’) can occur. This can take the form of proton tautomerism in compounds of formula (I) containing a cyclic urea, thiourea or cyanoguanidine group, or so-called valence tautomerism in compounds which contain an aromatic moiety. It follows that a single compound may exhibit more than one type of isomerism.

Included within the scope of the present invention are all stereoisomers, diastereoisomers (especially cis/trans isomers) and tautomeric forms of the compounds of formula (I), including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagent in place of the non-labelled reagent previously employed.

Pharmaceutically acceptable solvates in accordance with the invention include those wherein the solvent of crystallization may be isotopically substituted, e.g. D₂O, d₆-acetone, d₆-DMSO.

Also within the scope of the invention are intermediate compounds of formula (I) as hereinbefore defined, all salts, solvates and complexes thereof and all solvates and complexes of salts thereof as defined hereinbefore for compounds of formula (I). The invention includes all polymorphs of the aforementioned species and crystal habits thereof.

When preparing compounds of formula (I) in accordance with the invention, it is open to a person skilled in the art to routinely select the form of compound of formula (I) which provides the best combination of features for this purpose. Such features include the melting point, solubility, processability and yield of the intermediate form and the resulting ease with which the product may be purified on isolation.

The compounds of formula (I) should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

### Oral Administration

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulphate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

### Topical Administration

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm. Sci., 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

### Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

### Ocular/Aural Administration

The compounds of the invention may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Other Technologies

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in WO 91/11172, WO 94/02518 and WO 98/55148.

### Kit-of-Parts

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

### Dosage

For administration to human patients, the total daily dose of the compounds of the invention is typically in the range 1 mg to 1000 mg depending, of course, on the mode of administration. For example, oral administration may require a total daily dose of from 1 mg to 1000 mg, while an intravenous dose may require from 1 mg to 1000 mg. The total daily dose may be administered in single or divided doses and may, at the doctor's discretion, fall outside of the typical range given herein.

These dosages are based on an average human subject having a weight of about 60 kg to 70 kg. The doctor will readily be able to determine doses for subjects whose weight falls outside this range, such as infants and the elderly.

For the avoidance of doubt, references herein to “treatment” include references to curative, palliative and prophylactic treatment.

All of the compounds of formula (I) can be prepared by the procedures described in the General Methods described below or by the specific methods described in the Examples section and the Preparations section, or by routine modifications thereof. The present invention also encompasses any one or more of these processes for preparing the compounds of formula (I), in addition to any novel intermediates used therein.

### General Methods

The following abbreviations are used:

DMF=dimethylformamide
DMSO=dimethyl sulphoxide
THF=tetrahydrofuran
NMP=N-methyl-2-pyrrolidinone

### DMA=N,N-dimethylacetamide

DCM=dichloromethane
EDCI=1-(3-dimethylaminopropyl)-3-ethylcarbodiimide hydrochloride
CDI=1,1′-carbonyldiimidazole
TEMPO=2,2,6,6-tetramethylpiperidine-N-oxide

Compounds of formula (Ia′), which are compounds of formula (I) wherein A is O, B is a bond and R³ is (ii), may be prepared as shown in Scheme 1 below.

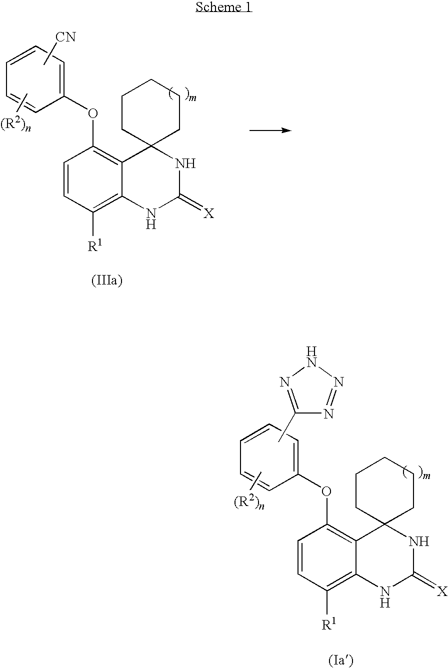

The compounds of formula (Ia′) may be prepared by reaction of compound (IIIa) with an azide such as trimethylsilyl azide in the presence of a catalyst such as dibutyltin oxide or with sodium azide and triethylamine in a suitable solvent such as toluene. Preferred conditions are: compound (IIIa), 2 eq trimethylsilyl azide and 0.1 eq dibutyltin oxide in toluene at 80° C. for 5 days adding further trimethylsilyl azide and dibutyltin oxide after each 24 hours.

Compounds of formula (IIIa) may be prepared as shown in Scheme 2 below.

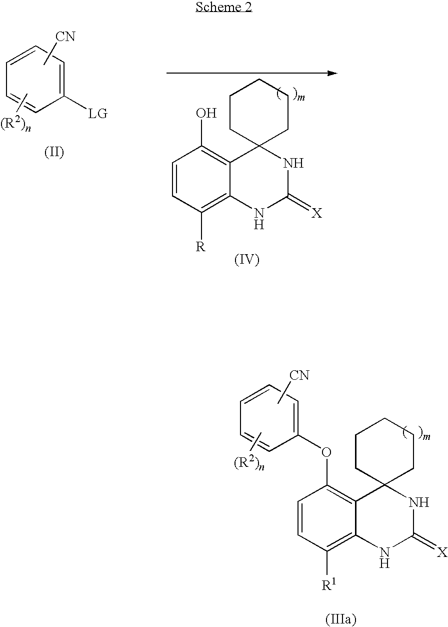

The compounds of formula (IIIa) may be prepared from compounds of formula (II) wherein LG is a suitable leaving group such as halogen, and a hydroxy compound of formula (IV) in a suitable solvent (eg DMF, DMSO) for 5-24 hours in the presence of a suitable base (eg Cs₂CO₃, K₂CO₃), at 50-120° C.

Preferred conditions are: 1 eq compound (1V), 1.1 eq compound (1I), 1.2 eq Cs₂CO₃, in DMF at 80° C. for 24 hours.

Compounds of formula (IV) are generally described in WO 02/074754. Specific compounds of formula (IV) wherein X is O, m is 1 and R¹ is Cl may be prepared as described in Bioorg. Med. Chem. Lett., (2004), 14 (18), 4627-32.

Compounds of formula (II) are available commercially or according to methods known to one skilled in the art.

Compounds of formula (Ia), which are compounds of formula (I) wherein B is a bond and R³ is (ii) may also be prepared from compounds of formula (III) in a two stage process as shown in Scheme 3.

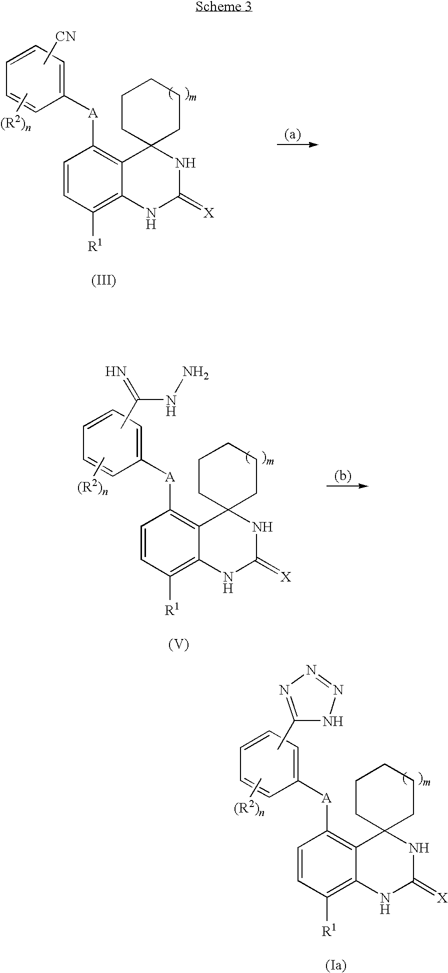

Compounds of formula (III) are generally described in WO 02/074754. Compounds of formula (III) wherein A is O may also be prepared as shown in Scheme 2 above. Compounds of formula (III) wherein A is a single bond, i.e. compounds of formula (X) wherein R^{b} is CN, may be prepared as shown in Scheme 7 below.

Step (a): Compounds of formula (V) may be prepared by reaction of compounds of formula (III) with hydrazine hydrate at 50-80° C. for 4-18 hours, optionally in the presence of phosphorus pentasulphide, in a solvent such as DMF, DMA or NMP.

Preferred conditions are: 1 eq compound (III), 2 eq hydrazine hydrate, 0.05 eq phosphorus pentasulphide in DMF at 70° C. for 18 hrs.

Step (b): The compounds of formula (Ia) may be prepared by the reaction of compounds of formula (V) with a nitrite (which may be inorganic, eg sodium nitrite, or organic, eg tert-butyl nitrite) in a suitable solvent such as acetic acid.

Preferred conditions are: 1 eq compound of formula (III) in acetic acid, 1.2 eq aqueous solution of sodium nitrite cautiously added at room temperature over 30 minutes.

Alternatively compounds of formula (Ia) may be prepared in a three stage process as shown in Scheme 4.

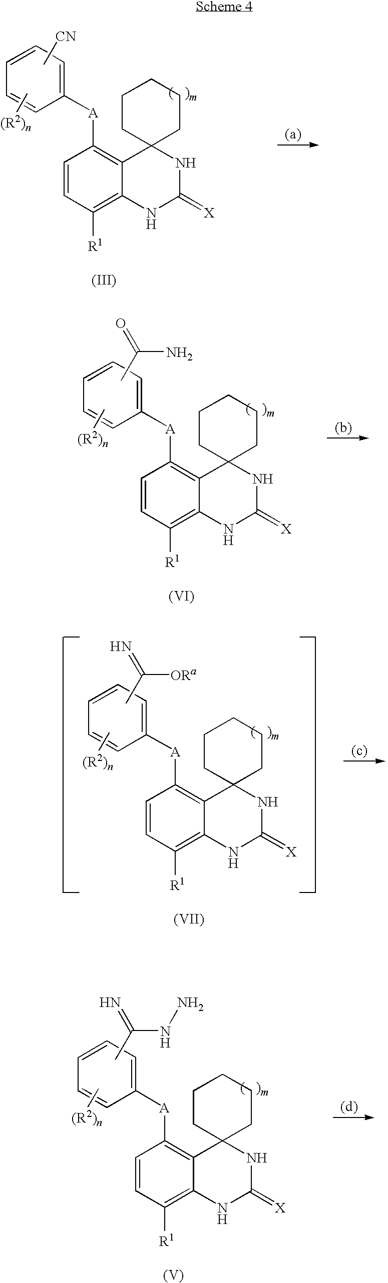

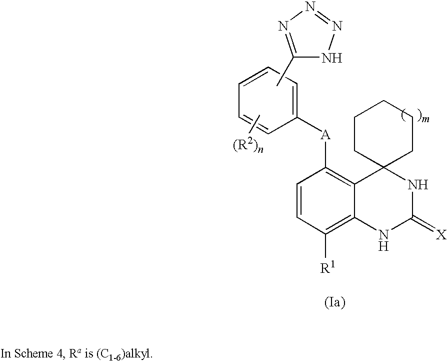

Step (a): The compounds of formula (III) may be hydrolysed under basic or acidic conditions, for example with aqueous hydrochloric or sulphuric acid in a suitable solvent such as 1,4-dioxane, acetic acid or ethanol or with an aqueous base such as lithium or sodium hydroxide with a suitable co-solvent such as 1,4-dioxane or ethanol at 90-120° C. for 6 to 24 hours.

Preferred conditions are: Compound (III) suspended in 3:1 concentrated sulphuric acid:water at 100° C. for 18 hours.

Step (b): Alkylation of compound (VI) with a compound of formula R^{a}-LG wherein LG is a leaving group (eg halogen, (C₁₋₆)alkyl-, benzene- or p-toluenesulphonyloxy, or di(C₁₋₆)alkyl ether), such as a tri(C₁₋₆)alkyloxonium salt, a (C₁₋₆)alkyl halide or a (C₁₋₆)alkyl p-toluenesulphonate, in the presence of a suitable base such as potassium or cesium carbonate in a solvent such as dichloromethane or DMF for 2-24 hours to yield an imidate of formula (VII) which is not isolated but used directly in step (c).

Preferred conditions are: 1 eq compound of formula (VI), 1.05 eq trimethyloxonium tetrafluoroborate in dichloromethane for 18 hours.

Step (c): The compounds of formula (V) may be prepared by treatment of the intermediate compound (VII) with hydrazine or a salt thereof in a solvent such as methanol or pyridine at room temperature for 1-18 hours.

Preferred conditions are: 1 eq compound of formula (VII), 3 eq hydrazine hydrate in methanol for 2 hours.

Step (d): The compounds of formula (Ia) may be prepared by the reaction of compound (V) with a nitrite (which may be inorganic, eg sodium nitrite, or organic, eg tert-butyl nitrite) under similar conditions to step (b) in Scheme 3.

Preferred conditions are: 1 eq compound of formula (III) in acetic acid, 1.2 eq aqueous solution of sodium nitrite cautiously added at room temperature over 30 minutes.

In a modification of Scheme 4, compounds of formula (VII) may be prepared directly from compounds of formula (III) by treatment with an alcohol of formula R^{a}OH, such as methanol or ethanol, in the presence of an acid such as hydrogen bromide or hydrogen chloride or a base such as potassium t-butoxide or sodium methoxide at 0° C. to room temperature for 6-24 hours.

Preferred conditions are: Compound of formula (III) in methanol saturated with gaseous hydrogen chloride at 0° C., warming to room temperature over 24 hours. Compounds of formula (Ib), which are compounds of formula (I) wherein R³ is (i), may be prepared as shown in Scheme 5 below.

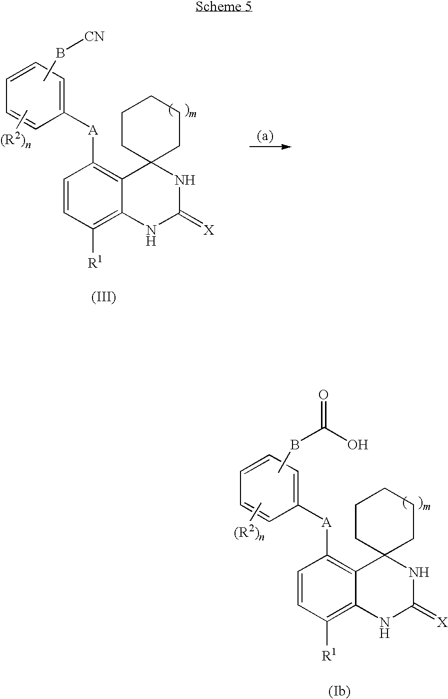

The compounds of formula (Ib) may be prepared from compounds of formula (III) by hydrolysis under acidic or basic conditions for example with aqueous hydrochloric or sulphuric acid in a suitable solvent such as 1,4-dioxane, acetic acid or ethanol or with an aqueous base such as lithium or sodium hydroxide with a suitable co-solvent such as 1,4-dioxane or ethanol at 90-120° C. for 6 to 24 hours.

Preferred conditions are: Compound (III) in a solution of equivalent volumes of concentrated sulphuric acid, acetic acid and water at 110° C. for 18 hours.

Compounds of formula (Ic), which are compounds of formula (I) wherein R³ is (vi) and R′ is (C₁₋₆)alkyl may be prepared as shown in Scheme 6, from the compounds of formula (Ib) by reaction with a (C₁₋₆)alkyl sulphonamide.

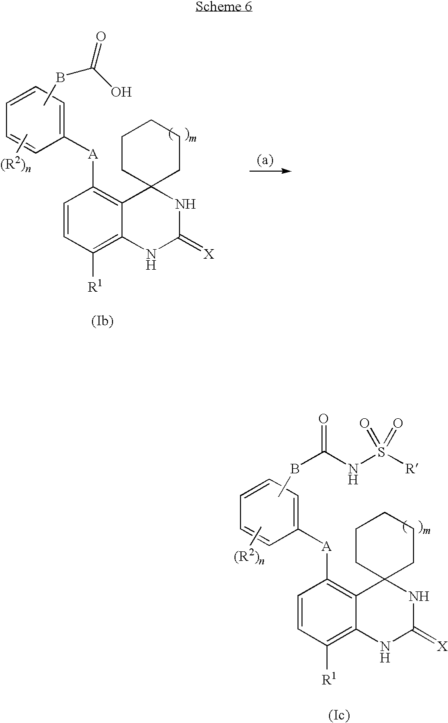

Step (a): The compounds of formula (Ic) may be prepared by coupling of the compounds of formula (Ib) with a (C₁₋₆)alkyl sulphonamide of formula R′SO₂NH₂. The acid is first activated by treatment with a suitable coupling reagent such as EDCI or CDI and then reacted with the sulphonamide in a suitable solvent such as DMF, THF or DCM.

Alternatively, compounds of formula (Ic) may be prepared by sulphonylation of a compound of formula (VI), shown in Scheme 4, with a (C₁₋₆)alkylsulphonyl halide or anhydride in the presence of a base such as sodium hydride, triethylamine or pyridine in a suitable solvent such as DCM, pyridine or THF.

Compounds of formula (Id), which are compounds of formula (I) wherein A is a single bond, B is a single bond and R^{b} is a group of the formula —CH₂OH, —CHO, —CN or —CO₂R^{b} wherein R^{c} is an ester residue (suitable examples of which are described in “Protective Groups in Organic Synthesis” (2^{nd} edition) by T. W. Greene and P. Wuts, Wiley and Sons, 1991; preferably (C₁₋₆)alkyl or benzyl) may be prepared as shown in Scheme 7.

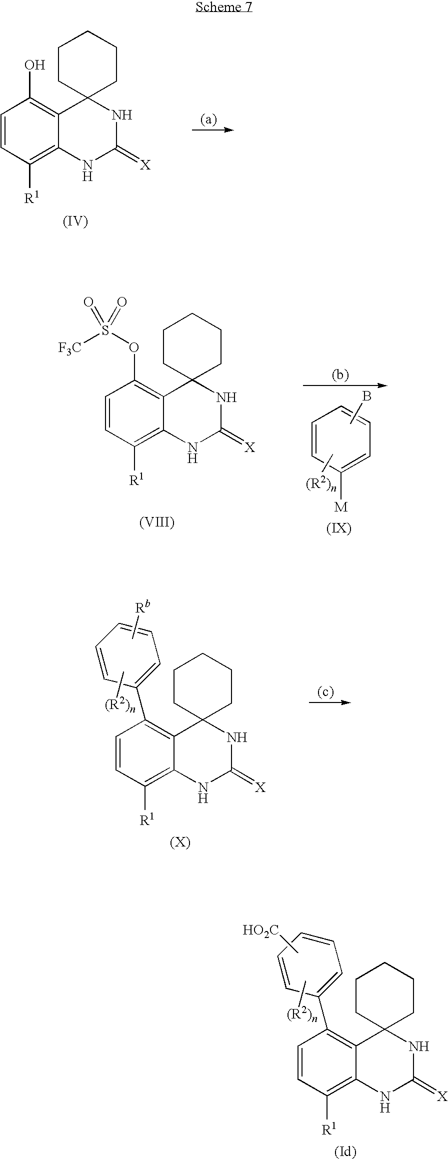

Step (a): The compounds of formula (VIII) may be prepared from compounds of formula (IV) by reaction with a compound of formula CF₃SO₂-LG, wherein LG is a leaving group such as halogen or CF₃SO₂O—, for example methanesulphonic anhydride or N-phenylbis(trifluoromethanesulphonimide), in the presence of a base such as triethylamine, pyridine or sodium hydride, in a suitable solvent such as THF, pyridine or dichloromethane at room temperature to 65° C.

Preferred conditions are: 1 eq compound (1V), 1 eq sodium hydride, 1.25 eq N-phenylbis(trifluoromethanesulphonimide), in THF at room temperature to 40° C. for 18 hours.

Step (b): Compounds of formula (X) may be prepared by cross-coupling compounds of formula (VIII) with compounds of formula (IX), where M is an optionally substituted metal or boron group suitable for cross-coupling, for example a tri(C₁₋₆)alkylstannane, boronic acid, pinacolatoboron or halozinc, in the presence of a suitable catalyst system, for example palladium tetrakis(triphenylphosphine), palladium acetate or palladium bis(dibenzylideneacetone), a base, for example sodium carbonate, potassium phosphate or cesium fluoride, in a suitable solvent such as toluene, 1,4-dioxane or dimethoxyethane at a temperature from 50° C. to 100° C.

Preferred conditions are: 1 eq compound (1V), 1.2 eq of compound (IX), 3.0 eq 2M aqueous Na₂CO₃ and 0.05 eq Pd(PPh₃)₄ in toluene:methanol 7:1 for 6 hours at 100° C.

Step (c): The compound of formula (Id) may be prepared by conversion of the functional group R^{b} of the compound of formula (X) to a carboxylic acid under known conditions for oxidation of an aldehyde or alcohol, or hydrolysis of a nitrile or ester.

Hydrolysis of a nitrile or ester may be achieved under acidic or basic conditions for example using aqueous hydrochloric or sulphuric acid in a suitable solvent such as 1,4-dioxane, acetic acid or ethanol or with an aqueous base such as lithium or sodium hydroxide with a suitable co-solvent such as 1,4-dioxane, or ethanol at 90-120° C. for 6 to 24 hours.

Preferred conditions wherein R^{b} is a nitrile or ester are: Compound (X) in a solution of equivalent volumes of concentrated sulphuric acid, acetic acid and water at 110° C. for 18 hours.

Additionally oxidation of an aldehyde or alcohol may be achieved with an oxidising agent in a suitable solvent. Typical reagents and conditions include catalytic chromium trioxide and periodic acid (H₅IO₆) in a solvent such as acetonitrile at room temperature to 50° C. for 18 to 36 hours, alternatively sodium hypochlorite plus sodium chlorite in the presence of catalytic TEMPO in a solvent such as acetonitrile at 0° C. to room temperature for 18 to 36 hours or sodium chlorite in the presence of 2-methyl-2-butene in aqueous THF.

Preferred conditions wherein R^{b} is an aldehyde are: 1 eq of Compound (X) in THF, 9 eq sodium chlorite, 7 eq sodium phosphate in water at room temperature for 18 hours.

Compounds of formula (Ie), which are compounds of formula (I) wherein R³ is (iii), may be prepared as shown in Scheme 8 below.

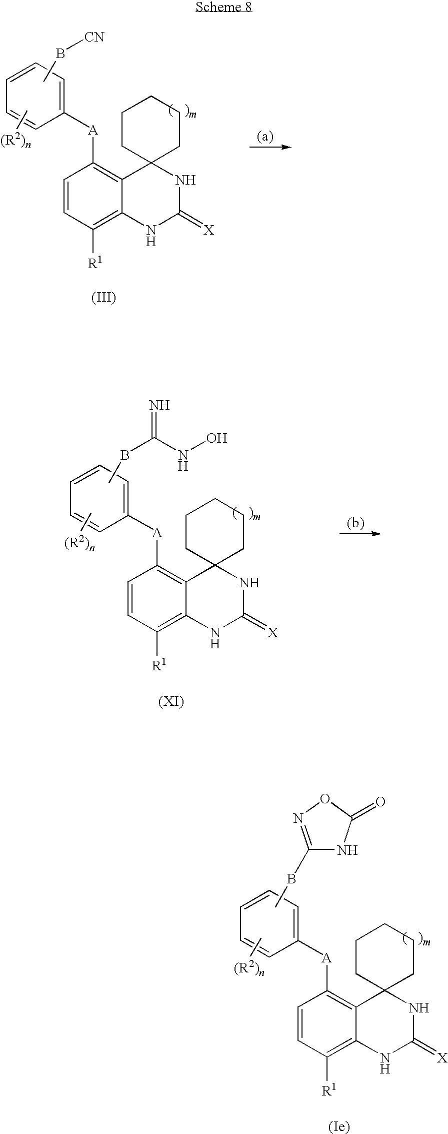

Step (a): The compounds of formula (XI) may be prepared by the reaction of a compound of formula (III) with hydroxylamine or a salt thereof, eg the hydrochloride, in the presence of a base such as sodium or potassium carbonate or a sodium or potassium (C₁₋₆)alkoxide, in a suitable solvent, for example methanol, ethanol or DMSO with or without additional water, at room temperature to 100° C. for 2-24 hours.

Preferred conditions are: 1 eq compound (III), 10 eq potassium tert-butoxide, 10 eq hydroxylamine hydrochloride in DMSO at 60° C. for 18 hours.

Step (b): The compounds of formula (Ie) may be prepared by reaction of an aldoxime of formula (XI) with a compound of formula LG-CO-LG (wherein LG is a suitable leaving group, such as halogen, (C₁₋₆)alkoxy or imidazole), for example carbonyl diimidazole, in a suitable solvent such as THF, DMF or 1,4-dioxane at 60-100° C. for 6-24 hours. Alternatively, reaction of compounds of formula (XI) with ethyl chloroformate in the presence of a base, for example potassium carbonate or pyridine, in a solvent such as acetone or DMF, at 0° C. to room temperature for 1-18 hours or diethylcarbonate, in the presence of a base such as sodium ethoxide or in a solvent such as ethanol at 0° C. to room temperature for 1-18 hours may be used to prepare compounds of formula (Ie).

Preferred conditions are: 1 eq compound (XI) and 1.2 eq carbonyl diimidazole in 1,4-dioxane at reflux for 2 hours, followed by 18 hours at room temperature.

Compounds of formula (If), which are compounds of formula (I) wherein R³ is (iv), may be prepared as shown in Scheme 9 below.

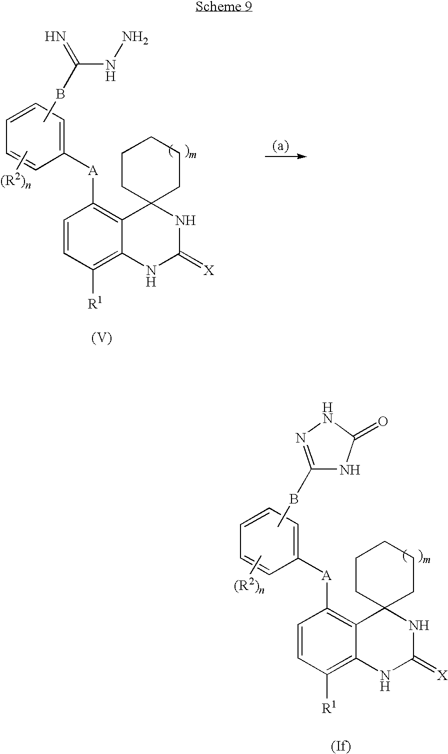

Step (a): Compounds of formula (If) may be prepared by reaction of a compound of formula (V) and a compound of formula LG-CO-LG under similar conditions to step (b) in Scheme 8.

Preferred conditions are: 1 eq compound (V) with 1.2 eq carbonyl diimidazole in 1,4-dioxane at 90° C. for 3 hours.

Compounds of formula (Ig), which are compounds of formula (I) wherein B is a bond and R³ is (vi), may be prepared as shown in Scheme 10.

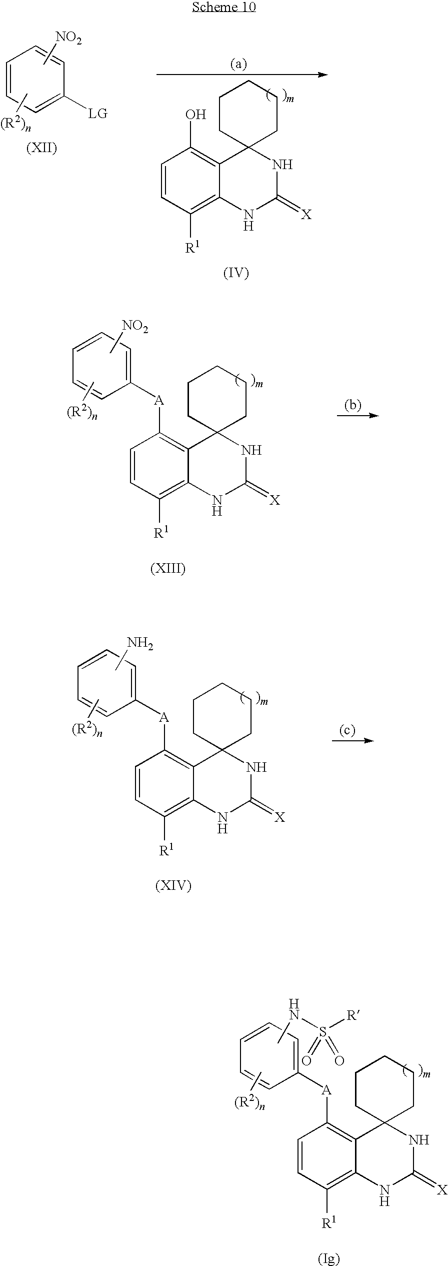

Step (a): The compounds of formula (XIII) may be prepared from compound (XII) and the hydroxy compound of formula (IV) in a suitable solvent (eg DMF, DMSO, acetone) in the presence of a suitable base such as cesium carbonate or potassium carbonate at room temperature to 100° C. for 5-24 hours.

Preferred conditions are: 1 eq compound (XII), 1 eq compound of formula (IV), 1.5 eq Cs₂CO₃ in DMF at room temperature for 18 hours.

Compounds of formula (XII) are available commercially or according to methods known to one skilled in the art.

Step (b): The compounds of formula (XIV) may be prepared by reduction of a compound of formula (XIII) under a variety of conditions which include hydrogenation with hydrogen or a transfer reagent such as ammonium formate and a suitable metal catalyst such as palladium or platinum on carbon. Alternative methods include reduction with a metal and an acid, typically iron or tin and acetic or hydrochloric acid, or sodium dithionite.

Preferred conditions are: 1 eq compound of formula (XIII), 5% by weight platinum on sulphided carbon in acetic acid at 1 atmosphere pressure of hydrogen at 50° C. for 18 hours.

Step (c): The compound of formula (Ig) may be prepared by reaction of the compound of formula (XIV) with a compound of formula R′SO₂-LG, wherein LG is a leaving group such as halogen or R′SO₂O—, for example trifluoro-methanesulphonyl chloride or anhydride, in the presence of a suitable base such as triethylamine or pyridine in a solvent such as DCM or THF at −78° C. to room temperature for 1-18 hours.

Preferred conditions are: 1 eq compound (XIV), 1 eq trifluoromethanesuiphonic anhydride, 1.5 eq triethylamine in DCM at −78° C. for 2 hours.

Compounds of formula (Ih), which are compounds of formula (I) wherein B is OCH₂ and R³ is (i), may be prepared as outlined in Scheme 11.

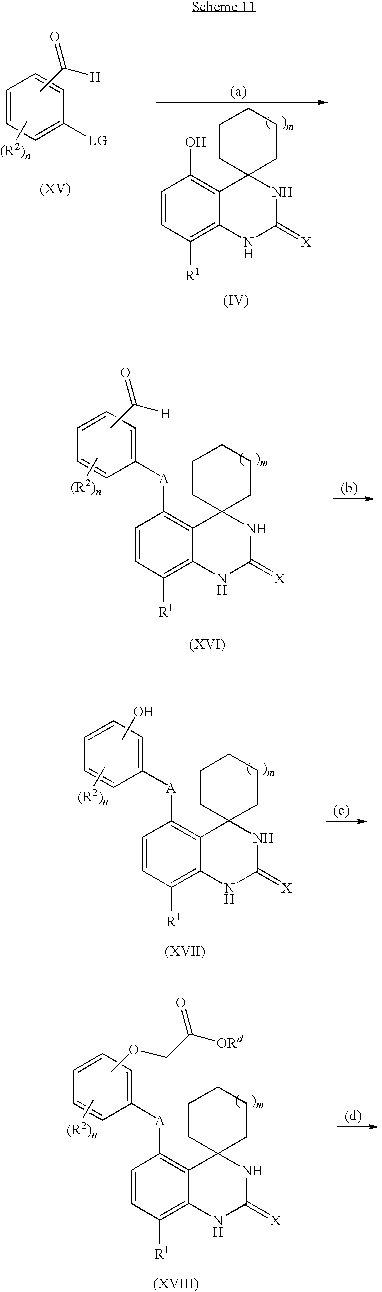

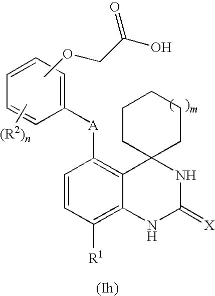

In Scheme 11, R^{d} is an ester residue, suitable examples of which are described in “Protective Groups in Organic Synthesis” (2^{nd} edition) by T. W. Greene and P. Wuts, Wiley and Sons, 1991. Preferably R^{d} is (C₁₋₆)alkyl or benzyl.

Step (a): The compounds of formula (XVI) wherein A is O may be prepared from compounds of formula (XV) in a similar manner to Scheme 2. Compounds of formula (XV) are available commercially or according to methods known to one skilled in the art.

Preferred conditions are: 1 eq compound (1V), 1.2 eq compound (XV), 1.5 eq Cs₂CO₃ in DMF at 70° C. for 24 hours.

Step (b): The compounds of formula (XVII) are typically prepared by Baeyer-Villiger oxidation of the compounds of formula (XVI) using for example hydrogen peroxide and acetic acid at 0-10° C. or 3-chloroperbenzoic acid in dichloromethane at room temperature for 6-18 hours.

Preferred conditions are: 1 eq compound (XVI), 3 eq 3-chloroperbenzoic acid in DCM at room temperature for 18 hours.

Alternatively, compounds of formula (XVII) wherein A is a bond may be prepared in a similar manner to step (b) of Scheme 7.

Step (c): Alkylation of a compound of formula (XVII) with a compound of formula LG-CH₂CO₂R^{d} (wherein LG is a leaving group, for example halogen), such as a suitably protected bromoacetate derivative, in the presence of a base such as potassium or cesium carbonate in a solvent such as DMF, THF or acetone at 50-90° C. for 2-18 hours give compounds of formula (XVIII).

Preferred conditions are: 1 eq compound (XVII), 1.2 eq bromoacetate, 1.2 eq cesium carbonate in DMF at 90° C. for 4 hours.

Step (d): The compounds of formula (XVII) may be hydrolysed to provide the compounds of formula (Ih). This reaction may be achieved under a variety of conditions, suitable examples of which are described in “Protective Groups in Organic Synthesis” (2^{nd} edition) by T. W. Greene and P. Wuts, Wiley and Sons, 1991. Preferred conditions are: Compound (XVIII) in a 3:1 mixture by volume of DCM: trifluoroacetic acid.

### Combinations

The PDE7 inhibitors of formula (I) may be usefully combined with another pharmacologically active compound, or with two or more other pharmacologically active compounds, particularly in the treatment of pain. For example, a PDE7 inhibitor of formula (I), or a pharmaceutically acceptable salt, solvate or prodrug thereof, as defined above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an Hᵢ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl)quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-c]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl-L-homocysteine, S42-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);
a sodium channel blocker, such as lidocaine;
a 5-HT3 antagonist, such as ondansetron;
   and the pharmaceutically acceptable salts and solvates thereof.

The ability of the compounds of formula (I) to inhibit PDE7 and PDE1 may be measured using the following assay protocol.

PDE7A, PDE7B and PDE1C enzymes catalyse the hydrolysis of 3′,5′-cyclic adenosine monophosphate (cAMP) to the 5′ adenosine monophosphate, 5′AMP. In a multiwell plate, PDE enzyme, [³H]-cAMP and the test compounds, are incubated at room temperature. The incubation is terminated by addition of commercially available yttrium silicate scintillation proximity assay (SPA) beads containing zinc sulphate. The yttrium silicate beads preferentially bind linear nucleotides, thus the product of the enzyme reaction, [³H]-5′AMP binds to the bead to produce a light signal, which is detected by a scintillation counter. The amount of signal produced directly correlates with the amount of product formed, and thus the activity of the enzyme. The maximum signal is obtained where enzyme and substrate are incubated alone. The background signal is measured from wells either containing no enzyme, or from wells containing a supra-maximal concentration of a known PDE 7A/7B/1C inhibitor. Each purified batch of enzyme is quality controlled and its Kₘ, Vₘₐₓ and specific activity determined from kinetic studies before use in compound inhibition studies. The inhibition of the enzyme, by a test compound, is calculated relative to the maximum and background responses. Using these data a % inhibition value is calculated relative to the maximum and minimum values obtained.

### Preparation of Working Solutions

A 1000 ml stock of buffer was prepared from the ingredients shown in Table 1 below:

TABLE 1

| | | Final | Stock Soln. | |
|---|---|---|---|---|
| | | concen- | concen- | |
| Reagent | Source | tration | tration | ml/1000 ml |
| HEPES (buffer) | Sigma | 50 mM | 1 | 50 |
| MgCl₂ | Sigma | 5 mM | 1 | 5 |
| Pluronic ® | Sigma | 0.025% | 5% | 5 |
| (detergent) | | | | |
| Millipore ® 18 mΩ | Millipore | | | 940 |
| purified water | | | | |

HEPES = 4-(2-hydroxyethyl)-1-piperazineethanesulfonic acid

The stock buffer was adjusted to pH 7.4 at room temperature and then filtered through a 0.2 μm filter. The stock buffer is stable at 4° C. for 1 month from the date of preparation.

On the day of experiment, Bovine Serum Albumin (BSA, available from Sigma) was added to the required volume of buffer to create a 0.00625% BSA final solution. This was achieved by preparing a stock 10% BSA solution as follows:

### Preparation of Stock 10% BSA Solution

1 g BSA was dissolved in 10 ml purified water, mixed by inversion to ensure homogeneity and aliquot in 100 μl volumes in appropriately labelled tubes. The 10% BSA solution is stable at −20° C. for up to 6 months.

An aliquot of the stock 10% BSA stock solution was removed from storage and allowed to thaw out at room temperature before being used to create the BSA working solution as shown in Table 2 below:

Preparation of 10 ml working BSA assay buffer

TABLE 2

| | | Final BSA |
|---|---|---|
| Reagent | Volume | concentration |
| 1x Buffer stock | 9.99 ml | |
| 10% BSA stock | 6.25 μl | 0.00625% |

### Preparation of Standard Compound and Controls

The compound of Example 75 of WO 02/074754, 5′-carboxypropoxy-8′-chloro-spiro[cyclohexane-1-4′-(3′,4′-dihydro)quinazolin]-2′(1′H)-one (referred to as compound A hereafter) was used as a standard for PDE7A and PDE7B. The compound of Example 32 of WO 02/074754, 4-(8′-chloro-2′-oxo-2′,3′-dihydro-1′H-spiro[cyclohexane-1,4′-quinazolin]-6′-yl)benzoic acid (referred to as compound B hereafter) was used as a standard for PDE1C.

4 mM stock solution prepared in 100% DMSO can be stored at 4° C. The volume of DMSO can be calculated as follows:

$$Volume of DMSO ( ml ) = \frac{weight of compound}{Molecular weight of compound} \times 250$$

The 30× Max control is a solution of 100% DMSO. The 30× Min control is achieved using a 30 μM of Compound A or B in 100% DMSO to yield no enzyme activity. 5 ml of a 30 μM solution of Compound A or B can be prepared by adding 4.962 ml of 100% DMSO to 37.5 μl of 4 mM Compound A or B.

### Method

On the day of assay, the 1× final assay buffer was prepared as detailed previously and kept on ice until needed.

### Kinetic Studies

For each new batch of enzyme, the Kₘ was determined, and the amount of enzyme required to obtain ˜1000 cpm signal in 45 minutes, whilst remaining in the linear portion of the reaction progress curve, was assessed. Ideally <10% of available [³H]-cAMP will be hydrolysed during the course of the assay.

### Enzyme Solution

The optimisation of this assay has been carried out using cell lysate containing full length PDE7A, PDE7B and PDE1C enzyme. The concentration of the enzyme in this cell lysate sample is unknown, so the specific activity of the cell lysate is used as a measure to ensure that the same activity per well is used despite any batch-to-batch variation of concentration/activity.

### Preparation of PDE7A/7B/1C Enzyme

PDE7A/7B/1C stock enzyme was prepared and kept at −20° C. in appropriately sized aliquots to reduce the number of freeze/thaw cycles. Table 3 below shows the volumes required to make 10 ml of PDE7A/7B/1C enzyme solution. PDE7A is diluted to 1/8000, PDE7B to 1/10000 and PDE1C to 1/200000.

TABLE 3

| | | Vol. of | | Overall |
|---|---|---|---|---|
| | | PDE stock/ | Vol. of | Dilution |
| | | diluted | Buffer + | of Enzyme |
| Enzyme | Dilution | soln (μl) | BSA (μl) | stock |
| PDE7A | PDE7B 1:100 | 5 | 495 | 1:100 |
| | dilution of stock | | | |
| | 1:40 dilution of | 250 | 9750 | 1:4000 |
| | above solution | | | |
| | This enzyme solution is further diluted when all the assay | | | |
| | components are dispensed into the assay plate i.e. 14 μl enzyme | | | |
| | solution is dispensed into a total assay volume of 30 μl, giving | | | |
| | an overall 1/8000-enzyme dilution. | | | |
| PDE7B | PDE7B 1:100 | 5 | 495 | 1:100 |
| | dilution of stock | | | |
| | 1:50 dilution of | 200 | 9800 | 1:5000 |
| | above solution | | | |
| | This enzyme solution is further diluted when all the assay | | | |
| | components are dispensed into the assay plate i.e. 14 μl enzyme | | | |
| | solution is dispensed into a total assay volume of 30 μl, giving | | | |
| | an overall 1/10000-enzyme dilution. | | | |
| PDE1C | PDE1C 1:10 | 50 | 450 | 1:10 |
| | dilution of stock | | | |
| | 1:10000 dilution of | 1 | 9999 | 1:100000 |
| | above solution | | | |

This enzyme solution is further diluted when all the assay
components are dispensed into the assay plate i.e. 14 μl enzyme
solution is dispensed into a total assay volume of 30 μl, giving
an overall 1/200000-enzyme dilution.

Once the enzyme solution was prepared it was kept on ice prior to usage

Preparation of 50 nM Adenosine 3′, 5′ Cyclic Phosphate (cAMP) Substrate Solution

The substrate is composed of a mixture of unlabelled cAMP and cAMP radiolabelled with tritium ([³H]-cAMP). The specifications of the stock of [³H]-cAMP will determine the volumes used.

The preparation of 9 ml of substrate solution using a [³H]-cAMP stock which is 1 mCi/ml and 24Ci/mmol (therefore 41.66 μM) is described below:

Kₘ for the enzymes batches to date is as follows:

| | | |
|---|---|---|
| PDE7A - 20 nM | PDE7B - 100 nM | PDE1C - 90 nM |

The assay requires 15 μl of substrate solution to be dispensed into a total assay volume of 30 μl, ie a ×2 dilution in the assay plate occurs.

### PDE7A/7B Substrate Solution

The final assay [cAMP] of ˜25 nM is required, so ˜50 nM [³H]-cAMP was prepared. 9 ml of substrate solution was prepared by mixing 10.8 μl of [³H]-cAMP (available from Amersham) with 8975 μl of assay buffer.

### PDE1C Substrate Solution

The final assay [cAMP]=75 nM therefore the required solution [cAMP]=0.15 μM (1/2 dilution in assay plate)

The desired [³H] per well=0.03 μCi, therefore the [³H] per μl of substrate solution=0.002 μCi (i.e. 0.03 μCi/16 μl of substrate solution per well).

Therefore for 9 ml the volume of [³H] required=18 μl (i.e. 9000 μl×0.002 μCi.

18 μl of this stock of [³H]-cAMP supplies 0.75 nmol [³H]-cAMP (i.e. 18 μl×0.042 nmol/μl), the total cAMP required to give the desired solution concentration is 1.35 nmol (i.e. 1.5×10⁻⁷ M×9×10⁻³ I=9×10⁻⁹ mol). Therefore 0.6 nmol of non-tritiated cAMP is required (i.e. 1.35 nmol-0.75 nmol).

The stock of cold cAMP is made to be 10 μM=10 nmol/ml, therefore 60 μl of this cold cAMP is required.

So to make 9 ml of substrate solution: 60 μl of cold cAMP+8922 μl of assay buffer+18 μl of [³H]-cAMP.

Volumes Required to Make 9 ml of cAMP Substrate Mix for PDE1C

TABLE 4

| | Final | | | | Volume of |
|---|---|---|---|---|---|
| | Concentration | | Concentration of | | stock |
| Reagent | required (μM/μCi) | | stock soln | | required (μl) |
| Unlabelled cAMP | 0.217 | μM | 10 | μM | 60 |
| Tritiated cAMP | 0.03 | μCi | 1 | mCi/ml | 18 |
| Buffer | | | | | 8922 |
| (supplemented | | | | | |
| with BSA) | | | | | |

For both PDE7A/7B and PDE1C substrate solutions, the exact concentration of cAMP was determined by taking 3 samples of 15 μl into scintillation vials. 4 ml Starscint® (a scintillation cocktail, available from Perkin Elmer), was then added and the tubes counted on a β-counter on a dpm program.

The concentration of radioligand is determined by the following equation:

$$[ Radioligand ] ( M ) = \frac{DPM}{\begin{matrix}\begin{matrix}( 2.22 \times {10}^{12} ) ( dpm \text{/} Ci ) \times \\ ( specific activity ) of radioligand ( Ci \text{/} Mol ) \times \end{matrix} \\ ( volume of sample ) counted ( L )\end{matrix}}$$

The concentration is then divided by 2 to allow for the ×2 dilution occurring in the assay plate.

Preparation of 6.6 mg/ml Yttrium Silicate PDE SPA Beads

Phosphodiesterase SPA beads (Yttrium Silicate) are available from Amersham.

Following the manufacturer's recommendations the vial of beads was reconstituted using 28 ml distilled or deionised water (˜20 mg/ml). The reconstituted beads are stable for 1 month when stored at 2-8° C. To prepare the beads for the assay, the reconstituted beads were diluted 3-fold in sterile double distilled water (˜6.6 mg/ml). The beads can settle, so were constantly stirred/agitated whilst dispensing.

30 μl of the ˜6.6 mg/ml beads are added to the 30 μl assay, giving a final bead concentration of ˜0.2 mg/well.

Compound dilutions and “background” wells were made 30 stronger than required in the assay plate to allow for 1 μl compound to be diluted by 29 μl of other assay components (14 μl enzyme and 15 μl radioligand). Thus for a final assay concentration of 10 μM, the compound must be at 300 μM in the compound addition plate. 4 mM stocks of compound are supplied in 100% DMSO (or are made up @ 4 mM from powder submissions). This requires 1/13.33 dilution in DMSO to be made.

### Assay Protocol

1 μl test compound was transferred into a suitable multi-well assay plate immediately prior to reagent assay addition, 14 μl enzyme solution was then added to the assay plate, followed by 15 μl substrate solution (ie: final assay volume 30 μl, with a final screening compound concentration of 1 μM for PDE7A and PDE7B and 10 μM for PDE1C). The plate was then sealed using a plate sealer and incubated at room temperature for 45 min on the plate shaker.

30 μl Yttrium Silicate PDE4 SPA beads were then added, ensuring constant stirring of the beads to give even distribution in the assay plate. The plate was then sealed using a plate sealer and incubated at room temperature for 30 mins on the plate shaker. The beads were then allowed to settle for 30 mins, before spinning the plates for 1 min at 200 g.

The plates were then read on a suitable radioactive counter, for example NXT-TopCount™ (available from Perkin Elmer) using the relevant protocol (30 second read time per well).

The data was fitted to a sigmoid curve using a least squares algorithm.

The IC₅₀ value was converted to a value using the Cheng-Prussof equation:

$${K}_{i} = \frac{{IC}_{50}}{1 + \frac{[ radioligand ]}{{K}_{m}}}$$

The PDE7 inhibitory activity of the compounds of Examples 1-25 was tested according to the above protocol. Compound C is 5-{[(8′-chloro-2′-oxo-2′,3′-dihydro-1′H-spiro[cyclohexane-1,4′-quinazolin]-5′-yl)oxy]methyl}-2-furoic acid, which is Example 80 of WO 02/074754; this document is considered the closest prior art to the compounds of the invention. The Kᵢ values obtained are shown in Table 5 below:

TABLE 5

| | PDE7A | PDE7B | PDE1C | 1C/7A | 1C/7B |
|---|---|---|---|---|---|
| | Kᵢ (nM) | Kᵢ (nM) | Kᵢ (nM) | selectivity | selectivity |
| Compound B | 1.5 | 39 | 1.4 | 0.97 | 0.04 |
| Compound C | 6.7 | 33 | 305 | 45 | 9.2 |
| Example 1 | 3.2 | 32 | >5600 | >1750 | >175 |
| Example 2 | 3.1 | 12 | >5630 | >1810 | >477 |
| Example 3 | 11 | 101 | 2430 | 218 | 24 |
| Example 4 | 7.5 | 45 | 1560 | 207 | 35 |
| Example 5 | 4.5 | 51 | >5730 | >1260 | >113 |
| Example 6 | 1.7 | 3.4 | 584 | 336 | 172 |
| Example 7 | 11 | 21 | 2260 | 201 | 107 |
| Example 8 | 4.8 | 9.4 | 1810 | 380 | 192 |
| Example 9 | 32 | 41 | >3750 | >116 | >92 |
| Example 10 | 0.1 | 0.4 | 73 | 743 | 186 |
| Example 11 | <0.2 | 1.0 | 70 | >307 | 70 |
| Example 12 | 0.3 | 0.7 | 89 | 301 | 123 |
| Example 13 | 1.7 | 7.2 | 862 | 523 | 119 |
| Example 14 | 2.4 | 4.4 | 723 | 301 | 165 |
| Example 15 | 5.0 | 6.6 | 1080 | 217 | 164 |
| Example 16 | 2.3 | 9.8 | 778 | 345 | 79 |
| Example 17 | <0.15 | 0.8 | 37 | >245 | 48 |
| Example 18 | 1.1 | 1.6 | 94 | 85 | 59 |
| Example 19 | 0.94 | 1.5 | 172 | 183 | 115 |
| Example 20 | 2.3 | 7.0 | 657 | 290 | 94 |
| Example 21 | 0.2 | 2.4 | 62 | 311 | 25 |
| Example 22 | <0.05 | 0.13 | 317 | >282 | 108 |
| Example 23 | 0.48 | 1.9 | 58 | 121 | 31 |
| Example 24 | 0.98 | 11 | 157 | 160 | 14 |
| Example 25 | NT | NT | NT | N/A | N/A |

NT = not tested

The data presented in Table 5 above shows a clear differentiation, with respect to PDE1C selectivity over PDE7A and/or PDE7B, between the compounds of Examples 1-25 of the present application and the closest prior art, Compounds B and C. This increased selectivity is likely to lead to the compounds exhibiting a decreased probability of cardiovascular toxicity in patients when compared with the closest prior art compounds.

The activity of a compound of formula (I) according to the present invention in the treatment of neuropathic pain may be measured according to the following test protocol.

Animals: Male Sprague Dawley rats (average weight 500 g) are housed in groups of 12. All animals are kept under a 12 h light/dark cycle (lights on at 07 h 00 min) with food and water ad libitum. All experiments are carried out by an observer blind to the treatments and in accordance with the Home Office Animals (Scientific Procedures) Act 1986.

### Chronic Constriction Injury (CCI) Rat Model of Neuropathic Pain

The CCI of sciatic nerve is performed as previously described (G. J. Bennett and Y. K. Xie, Pain (1988) 33, 87-107). Animals were anaesthetised with a 2% isofluorane/O₂ mixture. The right hind thigh is shaved and swabbed with 1% iodine. Animals are then transferred to a homeothermic blanket for the duration of the procedure and anaesthesia maintained during surgery via a nose cone. The skin is cut along the line of the thighbone. The common sciatic nerve is exposed at the middle of the thigh by blunt dissection through biceps femoris. About 7 mm of nerve is freed proximal to the sciatic trifurcation, by inserting forceps under the nerve and the nerve gently lifted out of the thigh. Suture is pulled under the nerve using forceps and tied in a simple knot until slight resistance is felt and then double knotted. The procedure is repeated until 4 ligatures (4-0 silk) are tied loosely around the nerve with approx 1 mm spacing. The incision is closed in layers and the wound treated with topical antibiotics.

### Streptozocin (STZ)—Induced Diabetes Neuropathy in the Rat

Diabetes is induced by a single intraperitoneal injection of streptozotocin (50 mg/kg) freshly dissolved in 0.9% sterile saline. Streptozotocin injection induces a reproducible mechanical allodynia within 3 weeks, lasting for at least 7 weeks (S. R. Chen and H. L. Pan. J. Neurophysiol. (2002), 87, 2726-2733).

### Assessment of Static and Dynamic Allodynia

### Static Allodynia

Animals are habituated to wire bottom test cages prior to the assessment of allodynia. Static allodynia is evaluated by application of von Frey hairs (Stoelting, Wood Dale, Ill., USA) in ascending order of force (0.6, 1, 1.4, 2, 4, 6, 8, 10, 15 and 26 grams) to the plantar surface of hind paws. Each von Frey hair is applied to the paw for a maximum of 6 seconds, or until a withdrawal response occurs. Once a withdrawal response to a von Frey hair is established, the paw is re-tested, starting with the filament below the one that produced a withdrawal, and subsequently with the remaining filaments in descending force sequence until no withdrawal occurred. The highest force of 26 g lifts the paw as well as eliciting a response, thus representing the cut off point. Each animal has both hind paws tested in this manner. The lowest amount of force required to elicit a response is recorded as paw withdrawal threshold (PWT) in grams. Static allodynia is defined as present if animals responded to a stimulus of, or less than, 4 g, which is innocuous in naive rats (M. J. Field et al. Pain (1999), 83, 303-11).

### Dynamic Allodynia

Dynamic allodynia is assessed by lightly stroking the plantar surface of the hind paw with a cotton bud. To avoid recording general motor activity, care is taken to perform this procedure in fully habituated rats that were not active. At least two measurements are taken at each time point, the mean of which represents the paw withdrawal latency (PWL). If no reaction is exhibited within 15 sec the procedure is terminated and animals are assigned this withdrawal time. A pain withdrawal response is often accompanied with repeated flinching or licking of the paw. Dynamic allodynia is considered to be present if animals respond to the cotton stimulus within 8 seconds of commencing stroking (Field et al, 1999, above).

### EXAMPLES

¹H Nuclear magnetic resonance (NMR) spectra were in all cases consistent with the proposed structures. Characteristic chemical shifts (8) are given in parts per million (ppm) downfield from tetramethylsilane using conventional abbreviations for designation of major peaks: e.g. s, singlet; d, doublet; t, triplet; q, quartet; m, multiplet; br, broad. The mass spectra (m/z) were recorded using either electrospray ionisation (ES or ESI) or atmospheric pressure chemical ionisation (AFCI). The following abbreviations have been used for common solvents: CDCl₃, deuterochloroform; D₆-DMSO, hexadeuterodimethylsulphoxide.

### Example 1

### 5-[(8′-chloro-2′-oxo-2′,3′-dihydro-1′H-spiro[cyclohexane-1,4′-quinazolin]-5′-yl)]-2-fluorobenzoic acid

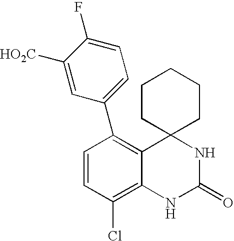

To a suspension of the compound from Preparation 2 (7.27 g, 19.6 mmol) in acetic acid (5 ml) was added sulphuric acid (5 ml) added followed by cautious addition of water (5 ml) and the resulting suspension heated at 120° C. for 6 hours. The reaction was cooled and water added to precipitate product. The solid was collected by filtration and washed well with water and air-dried to give crude product.

Recrystallisation from acetic acid/water (1:1, 240 ml) afforded the title compound as an off-white solid (6.74 g, 17.3 mmol, 88%) after drying in vacuo at 50° C.

¹H-NMR (DMSO-d₆, 400 MHz): δ 0.60-1.69 (m, 10H), 6.64 (d, 1H), 6.78 (s, 1H), 7.34 (m, 2H), 7.51 (m, 1H), 7.63 (m, 1H), 8.38 (s, 1H).

LRMS m/z (ESI) 389 [M+H]⁺

### Example 2

### 3-(8′-chloro-2-oxo-2′,3′-dihydro-1′H-spiro[cyclohexane-1,4′-quinazolin]-5′-ylbenzoic acid

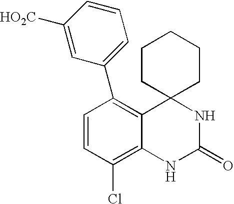

The title compound (16 mg, 0.043 mmol, 78%) was prepared in a similar manner to Example 1 starting with the compound from Preparation 3 (21 mg, 0.055 mmol).

¹H-NMR (DMSO-d₆, 400 MHz): δ 1.18-1.64 (m, 10H), 6.63 (d, 1H), 6.77 (d, 1H), 7.33 (d, 1H), 7.51 (t, 1H), 7.54 (d, 1H), 7.73 (s, 1H), 7.96 (d, 1H), 8.36 (s, 1H).

LRMS m/z (ESI) 371 [M+H]⁺

### Example 3

### 5-[(8′-chloro-2′-oxo-2′,3′-dihydro-1′H-spiro[cyclohexane-1,4′-quinazolin]-4′-yl)]-2-fluorobenzoic acid

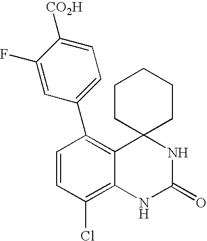

To a suspension of the product of Preparation 4 (20 mg, 0.054 mmol) in t-butanol (5 ml) was added 2M 2-methyl-2-butene in THF (0.27 ml, 0.54 mmol) followed by dropwise addition of a solution of sodium chlorite (56 mg, 0.494 mmol) and sodium phosphate (51 mg, 0.37 mmol) in water (2 ml). The reaction mixture turned to a yellow solution and was stirred at room temperature for 18 hours. The reaction mixture was diluted with water (5 ml), acidified with 2M aqueous HCl and extracted with ethyl acetate (10 ml). The organic layer was washed with brine, dried (MgSO₄) and concentrated in vacuo to give a solid residue. Trituration with diethyl ether afforded the title compound as an off-white solid (5.6 mg, 0.014 mmol, 27%).

¹H-NMR (DMSO-d₆, 400 MHz) δ1.09-1.68 (10H, m), 6.59 (1H, d), 6.83 (1H, broad s), 7.17 (1H, dd), 7.19 (1H, d), 7.24 (1H m), 7.33 (1H, d), 7.85 (1H, t), 8.45 (1H, broad s).

### Example 4

### 8′-chloro-5-[4-fluoro-3-(2H-tetrazol-5-yl)phenyl]-1′H-spiro[cyclohexane-1,4′-quinazolin]-2′(3′H)-one

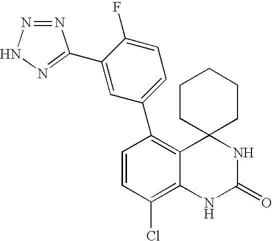

To a suspension of the product from Preparation 2 (97 mg, 0.26 mmol) in toluene (5 ml) under nitrogen atmosphere at room temperature was added azidotrimethylsilane (0.14 ml, 1.05 mmol) followed by dibutyltin (IV) oxide (6.5 mg, 0.026 mmol). The slurry was heated to 110° C. for 18 hours, another portion of azidotrimethylsilane (0.07 ml, 0.5 mmol) and dibutyltin(IV) oxide (6.5 mg, 0.026 mmol) were added and heating continued for 24 hours. This was repeated once more and the reaction was then concentrated under reduced pressure. 2N aqueous HCl was added, followed by addition of acetone to give a solid. The product was collected by filtration to give the title compound as a white solid (61 mg, 0.14 mmol, 57%).

¹H-NMR (DMSO-d₆, 400 MHz) δ 0.64-1.68 (m, 10H), 6.66 (d, 1H), 6.84 (broad s, 1H), 7.37 (d, 1H), 7.54 (m, 2H), 7.86 (d, 1H), 8.45 (broad s, 1H).

LRMS m/z (ESI) 413 [M+H]⁺

### Example 5

### (a) Ethyl [3-(8′-chloro-2′-oxo-2′,3′-dihydro-1′H-spiro[cyclohexane-1,4′-quinazolin]-5′-yl)phenoxy]acetate

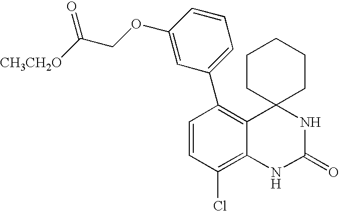

The product from Preparation 5 (250 mg, 0.73 mmol), Cs₂CO₃ (285 mg, 0.875 mmol) and ethyl bromoacetate (80 μl, 11.73 mmol) were combined in DMF (2 ml) and heated at 90° C. for 6 hours and allowed to cool to room temperature overnight. Further ethyl bromoacetate (8 μl, 0.1 eq) was added and the mixture heated at 90° C. for 6 hours. The reaction was quenched by the addition of water and allowed to cool to room temperature overnight. The white solid was collected by filtration and air-dried to give 230 mg crude product which was recrystallised from acetic acid:water to give the title compound after drying in vacuo at 50° C. as a white solid (204 mg, 0.475 mmol, 65%).

¹H-NMR (DMSO-d₆, 400 MHz) δ 0.71 (m, 1H), 1.14 (t, 3H), 1.20-1.46 (complex, 5H), 1.55 (m, 4H), 4.10 (q, 2H), 4.76 (s, 2H), 6.56 (d, 1H), 6.72 (m, 2H), 6.80 (d, 1H), 6.92 (d, 1H), 7.26 (m, 2H), 8.27 (s, 1H).

LRMS m/z (ESI) 429[M+H]⁺

### (b) [3-(8′-chloro-2′-oxo-2′,3′-dihydro-1′H-spiro[cyclohexane-1,4′-quinazolin]-5′-yl)phenoxy]acetic acid

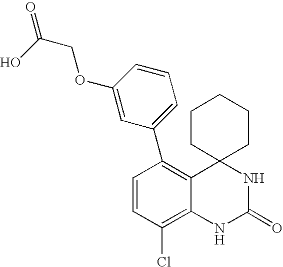

The product of step (a) (204 mg, 0.47 mmol) was suspended in methanol and 2M aqueous NaOH (0.48 ml, 0.95 mmol) added and the mixture heated at 50° C. for 2 hours then stood at room temperature overnight (convenience). Additional 2M NaOH (1 ml) and water (15 ml) were added and the solution extracted with ethyl acetate (2×15 ml). The aqueous solution was acidified with 2N HCl to pH 2 at which point a white solid precipitated. The solid was collected by filtration, washed well with water and dried in vacuo to give the title compound as a white solid (131 mg, 0.326 mmol, 68%).

¹H-NMR (DMSO-d₆, 400 MHz) δ 0.74 (m, 1H), 1.32 (complex, 4H), 1.41 (m, 1H), 1.58 (m, 4H), 4.68 (s, 2H), 6.58 (d, 1H), 6.73 (m, 1H), 6.76 (m, 1H), 6.80 (d, 1H), 6.92 (dd, 1H), 7.29 (m, 2H), 8.36 (s, 1H), 12.98 (broad, 1H).

LRMS m/z (ESI) 399[M−H]⁺

### Example 6

### 2-{(8′-chloro-2′-oxo-2′,3′-dihydro-1′H-spiro[cyclohexane-1,4′-quinazolin]-5′-yl)oxy}-3-fluorobenzoic acid

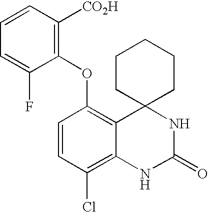

The compound of Preparation 6 (20 g, 52 mmol) was suspended in acetic acid (100 ml) and heated to 60° C. before adding water (100 ml) and sulphuric acid (100 ml) and the reaction was stirred at 110° C. for 18 hours. The resulting suspension was allowed to slowly cool to room temperature. The crystalline product was collected by filtration, washed with water and air-dried to afford an off-white solid (approx 19 g) which was recrystallised in acetic acid:water (9:1, ˜300 ml) to give the title compound as a white solid, (17.3 g, 42.7 mmol, 82%).

¹H-NMR (DMSO-d₆, 400 MHz). [symbol] 1.10 (m, 1H), 1.59 (m, 1H), 1.87-1.63 (m, 4H) 2.44 (m, 2H), 2.57 (broad m, 2H), 6.00 (d, 1H), 7.09 (s, 1H), 7.16 (d, 1H), 7.42 (m, 1H), 7.71 (d, 1H), 8.18 (s, NH).

LRMS m/z (ESI) 405 [M+H]⁺

Elemental analysis:

Calculated: C=59.34%, H=4.48%, N=6.92%;

Observed: C=59.28%, H=4.44%, N=6.83%.

### Example 7

### 2-{(8′-chloro-2′-oxo-2′,3′-dihydro-1′H-spiro[cyclopentane-1,4′-quinazolin]-5′-oxy}-3-fluorobenzoic acid

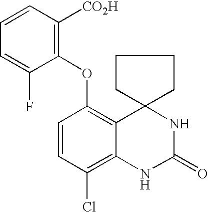

The title compound (1.38 g, not dry, quantitative) was prepared in a similar manner to Example 6 starting with the product of Preparation 8 (1.25 g, 3.36 mmol).

¹H-NMR (DMSO-d₆, 400 MHz): δ 1.61 (m, 2H), 1.79 (m, 4H), 2.43 (m, 1H), 2.64 (m, 1H), 5.98 (d, 1H), 7.15 (d, 1H), 7.20 (m, 1H+NH), 7.62 (t, 1H), 7.71 (d, 1H), 8.15 (NH, 1H).

LRMS m/z (ESI) 391[M+H]⁺

### Example 8

### 3-chloro-2-{(8′-chloro-2′-oxo-2′,3′-dihydro-1′H-spiro[cyclohexane-1,4′-quinazolin]-5′-yl)oxy}benzoic acid

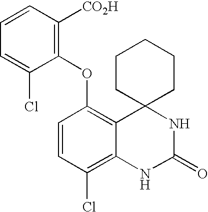

The title compound (530 mg, 1.25 mmol, 92%) was prepared in a similar manner to Example 6 starting with the compound of Preparation 9 (550 mg, 1.37 mmol).

¹H-NMR (DMSO-d₆, 400 MHz): [symbol] 1.11 (m, 1H), 1.46 (m, 2H), 1.59 (m, 1H), 1.62-1.85 (m, 4H), 2.64 (m, 2H), 5.81 (d, 1H), 7.03 (NH, 1H), 7.13 (d, 1H), 7.42 (t, 1H), 7.84 (m, 2H), 8.09 (NH, 1H).

LRMS m/z (ESI) 421[M+H]⁺

### Example 9

### 3-chloro-2-{(8′-fluoro-2′-oxo-2′,3′-dihydro-1′H-spiro[cyclohexane-1,4′-quinazolin]-5′-yl)oxy}benzoic acid

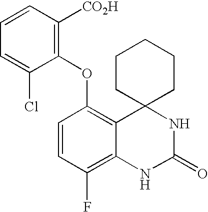

The title compound (110 mg, 0.27 mmol, 75%) was prepared in a similar manner to Example 6 starting with the product of Preparation 10 (140 mg, 0.36 mmol).

¹H-NMR (DMSO-d₆, 400 MHz): δ 1.10 (m, 1H), 1.45 (m, 2H), 1.59 (m, 1H), 1.64-1.86 (m, 4H), 2.64 (m, 2H), 5.66 (m, 1H), 6.82 (NH, 1H), 6.87 (t, 1H), 7.41 (m, 1H), 7.81 (m, 2H), 8.99 (NH, 1H).

LRMS m/z (ESI) 405[M+H]⁺

### Example 10

### 8′-chloro-5′-[2-fluoro-6-(2H-tetrazol-5-yl)phenoxy]-1′H-spiro[cyclohexane-1,4′-quinazolin]-2′(3′H)-one

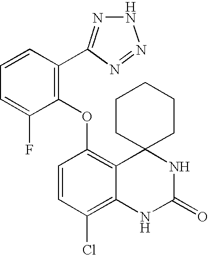

To a solution of the crude product from Preparation 22 (˜19.3 g, 46.2 mmol) in acetic acid (800 ml) was added a solution of sodium nitrite (3.82 g, 55.4 mmol) in water (200 ml) dropwise over 30 minutes at room temperature. The initially yellow solution darkened to orange immediately. The reaction was complete at the end of the addition. Water (˜1000 ml) was added slowly with vigorous stirring to precipitate a pale yellow solid which was left to stir for 18 hours and then collected by filtration. This solid was suspended in 17% aqueous ammonia (800 ml) and stirred for 30 minutes before adding ethyl acetate (100 ml); addition of saturated brine was required to effect separation of the phases. The organic layer was removed and the aqueous was washed a second time with ethyl acetate (100 ml). The aqueous layer was then slowly added to a stirred solution of 6M HCl (˜2000 ml) and allowed to stir for 18 hours to precipitate an off white solid. The product was collected by filtration then recrystallised from acetic acid:water. Drying in vacuo gave the title compound as an off-white solid (9.2 g, 21.4 mmol, 46%).

¹H-NMR (DMSO-d₆, 400 MHz) δ 0.99-1.11 (m, 1H), 1.35-1.46 (m, 2H), 1.53-1.62 (m, 2H), 1.70-1.82 (m, 3H), 2.31-2.40 (m, 1H), 2.49-2.55 (m, 1H), 6.07 (d, 1H), 7.07 (s, 1H), 7.51-7.56 (m, 1H), 7.62-7.67 (m, 1H), 7.81 (d, 1H), 8.19 (s, 1H).

LRMS m/z (APCI) 429 [M+H]⁺, (ESI) 429 [M+H]⁺

### Example 11

### 8′-chloro-5′-[4-fluoro-2-(1H-tetrazol-5-yl)phenoxy]-1′H-spiro[cyclohexane-1,4′-quinazolin]-2′(3′H)-one

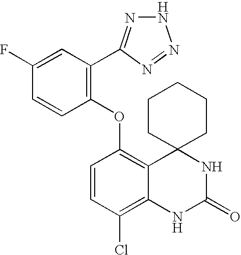

The title compound (3.97 g, 9.23 mmol, 64%) was prepared in a similar manner to Example 10 starting with the product of Preparation 23 (6.0 g, 14.4 mmol).

¹H-NMR (400 MHz, DMSO-d₆) δ 0.76 (m, 1H), 1.29 (m, 2H), 1.46 (m, 1H), 1.68 (m, 4H), 2.10 (broad, 2H), 6.48 (d, 1H), 7.03 (s, 1H), 7.08 (m, 1H), 7.31 (d, 1H), 7.42 (m, 1H), 7.83 (m, 1H), 8.28 (s, 1H).

LRMS m/z (APCI) 429 [M+H]⁺

### Example 12

### 8′-chloro-5′-[6-fluoro-2-(1H-tetrazol-5-yl)phenoxy]-1′H-spiro[cyclohexane-1,4′-quinazolin]-2′(3′H)-one

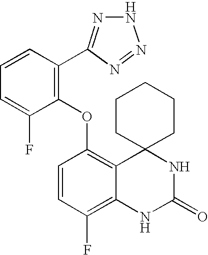

The title compound (120 mg, 0.29 mmol, 77%) was prepared in a similar manner to Example 10 starting with the product of Preparation 24 (150 mg, 0.374 mmol).

¹H-NMR (DMSO-d₆, 400 MHz) δ 1.18 (m, 1H), 1.41 (m, 2H), 1.58 (m, 2H), 1.79 (m, 4H), 2.37 (m, 1H), 5.99 (m, 1H), 6.96 (m, 2H), 7.55 (m, 1H), 7.65 (m, 1H), 7.80 (m, 1H), 9.03 (s, 1H).

LRMS m/z (APCI) 413 [M+H]⁺

### Example 13

### 8′-chloro-5′-[4-fluoro-2-(1H-tetrazol-5-yl)phenoxy]-1′H-spiro[cyclopentane-1,4′-quinazolin]-2′(3′H)-one

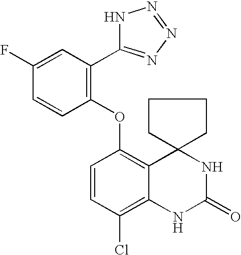

The title compound (430 mg, 1.03 mmol, 26%) was prepared in a similar manner to Example 10 starting with the product of Preparation 25 (1.6 g, 3.96 mmol).

¹H-NMR (400 MHz, DMSO-d₆) δ 1.24 (m, 2H), 1.61 (m, 2H), 1.70 (m, 2H), 1.98-2.40 (poorly resolved m, assumed 2H), 6.41 (d, 1H), 7.16 (m, 1H), 7.25 (m, 1H), 7.34 (s, NH), 7.42 (m, 1H), 7.82 (m, 1H), 8.17 (s, NH).

LRMS m/z (ESI) 415 [M+H]⁺

### Example 14

### 8′-chloro-5′-[6-fluoro-2-(1H-tetrazol-5-yl)phenoxy]-1′H-spiro[cyclopentane-1,4′-quinazolin]-2′(3′H)-one

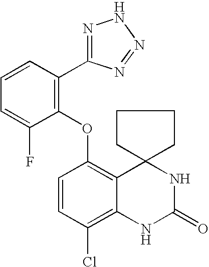

The title compound (12.115 g, 29.2 mmol, 41%) was prepared in a similar manner to Example 10 starting with the product of Preparation 26 (28.4 g, 70.41 mmol).

¹H-NMR (DMSO-d₆, 400 MHz) δ 1.42-1.60 (m, 2H), 1.65-1.84 (m, 4H), 2.39-2.48 (m, 2H), 6.05 (d, 1H), 7.12 (d, 1H), 7.37 (s, 1H), 7.51-7.56 (m, 1H), 7.81-7.83 (m, 1H), 7.82 (d, 1H), 8.15 (s, 1H).

LRMS (APCI) 415 [M+H]⁺

### Example 15

### 8′-chloro-5′-[6-chloro-2-(1H-tetrazol-5-yl)phenoxy]-1′H-spiro[cyclopentane-1,4′-quinazolin]-2′(3′H)-one

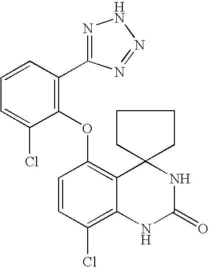

The title compound (2.2 g, 5.1 mmol, 32%) was prepared in a similar manner to Example 10 starting with the product of Preparation 27 (6.68 g, 15.9 mmol).

¹H-NMR (DMSO-d₆, 400 MHz) [symbol] 1.43 (m, 6H), 2.4-2.5 (m, 1H), 2.65 (m, 1H), 5.84 (d, 1H), 7.08 (d, 1H), 7.36 (s, 1H), 7.56 (m, 1H), 7.87 (m, 1H), 7.96 (d, 1H), 8.15 (s, 1H).

LRMS m/z (APCI) 431 [M+H]⁺

### Example 16

### 8′-chloro-5′-[2-(1H-tetrazol-5-yl)phenoxy]-1′H-spiro[cyclopentane-1,4′-quinazolin]-2′(3′H)-one

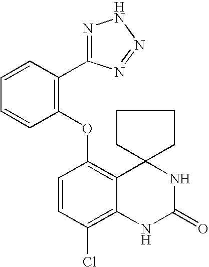

The title compound (396 mg, 0.96 mmol, 47%) was prepared in a similar manner to Example 10 starting with the product of Preparation 29 (800 mg, 2.07 mmol).

¹H-NMR (DMSO-d₆, 400 MHz) δ 1.11-1.24 (m, 2H), 1.1.51-1.62 (m, 2H), 1.67-1.75 (m, 2H), 1.92-2.34 (broad s, 2H), 6.50 (s, 1H), 7.06 (d, 1H), 7.29-7.35 (m, 3H), 7.53-7.58 (m, 1H), 7.98-8.00 (m, 1H), 8.27 (s, 1H).

LRMS m/z (ESI) 397 [M+H]⁺

### Example 17

### 8′-chloro-5′-[2-(1H-tetrazol-5-yl)phenoxy]-1′H-spiro[cyclohexane-1,4′-quinazolin]-2′(3′H)-one

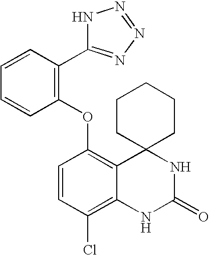

The title compound (2.3 g, 5.59 mmol, 59%) was prepared in a similar manner to Example 10 starting with the product of Preparation 31 (3.77 g, 9.43 mmol).

¹H-NMR (DMSO-d₆, 400 MHz) δ 0.64-0.75 (d, 1H), 1.25 (d, 2H), 1.43 (d, 1H), 1.60-1.72 (m, 4H), 2.03 (broad s, 2H), 6.54 (d, 1H), 6.98 (d, 1H), 7.02 (s, 1H), 7.30-7.35 (m, 2H), 7.53-7.57 (m, 1H), 7.99 (m, 1H), 8.29 (s, 1H).

LRMS m/z (APCI) 411[M+H]⁺

### Example 18

### 8′-chloro-5′-[2-fluoro-6-(5-oxo-4,5-dihydro-1,2,4-oxadiazol-3-yl)phenoxy]-1′H-spiro[cyclohexane-1,4′-quinazolin]-2′(3′H)-one

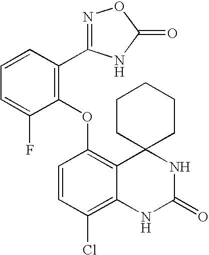

The product of Preparation 37 (45 mg, 0.11 mmol) was suspended in 1,4-dioxane (1 ml). 1,1′-carbonyldiimidazole (21 mg, 0.129 mmol) was added and the mixture heated at 120° C. for 2 hours then allowed to cool to room temperature over 18 hours. 2N HCl added and the mixture stirred for 30 minutes. The resulting solid was collected by filtration, washed with water and dried in vacuo to give the title compound as a slightly grey powder (35 mg, 0.078 mmol, 73%).

¹H-NMR (CD₃OD, 400 MHz) δ 1.25-1.36 (m, 1H), 1.61-1.78 (m, 5H), 1.84-1.93 (m, 2H), 2.57-2.71 (m, 2H), 6.16 (d, 1H), 7.15 (d, 1H), 7.47-7.58 (m, 3H)

LRMS m/z (APCI) 445 [M+H]⁺

### Example 19

### 8′-chloro-5′-[2-fluoro-6-(5-oxo-4,5-dihydro-1H-1,2,4-triazol-3-yl)phenoxy]-1′H-spiro[cyclohexane-1,4′-quinazolin]-2′(3′H)-one

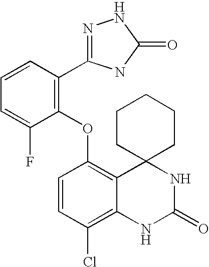

The product of Preparation 22 (100 mg, 0.239 mmol) was heated in 1,4-dioxane (1 ml) in oil bath at 90° C. to dissolve. 1,1′-carbonyldiimidazole (47 mg, 0.287 mmol) was added and the suspension heated at 90° C. for 3 hours and then allowed to cool over 18 hours. 2N HCl was added to the pale pink suspension and stirred for 2 hours. The resulting solid was collected by filtration and dried in vacuo to give the title compound as a pale pink solid (80 mg, 0.18 mmol, 63%).

¹H-NMR (DMSO-d₆, 400 MHz) δ 1.08-1.18 (m, 1H), 1.47 (d, 2H), 1.58 (d, 1H), 1.66-1.84 (m, 4H), 2.53-2.59 (m, 2H), 6.01 (d, 1H), 7.05 (s, 1H), 7.15 (d, 1H), 7.44-7.58 (m, 3H), 8.11 (s, 1H), 11.65 (s, 1H), 11.83 (s, 1H)

LRMS m/z (APCI) 442 [M−H]⁻

### Example 20

### 2-[(8′-chloro-2′-oxo-2′,3′-dihydro-1′H-spiro[cyclohexane-1,4′-quinazolin]-5′-yl)oxy]-3-fluoro-N-(methylsulfonyl)benzamide

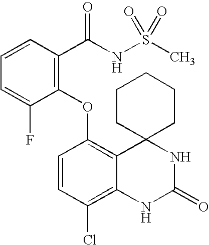

The product of Example 7 (100 mg, 0.243 mmol), DMAP (89 mg, 0.73 mmol) and methanesulfonamide (70 mg, 0.73 mmol) were suspended in DMF (2 ml) and EDCI (140 mg, 0.73 mmol) was added. The mixture was stirred at room temperature for 18 hours after which 2N HCl added to precipitate a solid. The solid was collected by filtration, washed with water and then dried in vacuo to give the title compound as an off-white solid (98 mg, 0.203 mmol, 84%).

¹H-NMR (400 MHz, DMSO-d₆) δ 1.17-1.27 (m, 1H), 1.47 (d, 2H), 1.59 (d, 1H), 1.67-1.85 (m, 4H), 2.44-2.54 (2H masked by DMSO), 3.18 (s, 3H), 6.10 (d, 1H), 7.08 (s, 1H), 7.21 (d, 1H), 7.41-7.46 (m, 1H), 7.49-7.51 (m, 1H), 7.57-7.61 (m, 1H), 8.17 (s, 1H), 12.32 (broad s, 1H).

LRMS m/z (APCI) 482 [M+H]⁺

### Example 21

### N-{2-[(8′-chloro-2′-oxo-2′,3′-dihydro-1′H-spiro[cyclohexane-1,4′-quinazolin]-5′-yl)oxy]-3-fluorophenyl}-1,1,1-trifluoromethanesulfonamide

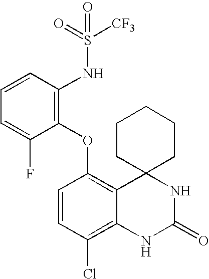

The product of Preparation 19 (70 mg, 0.19 mmol) was dissolved in DCM and triethylamine (32 μl, 0.224 mmol) added. The mixture was cooled to −78° C. then trifluoromethanesulphonic anhydride (32 μl, 0.19 mmol) added in one portion. The mixture was kept at −78° C. for 2 hours then warmed to room temperature, water added and extracted into ethyl acetate. The product was purified by chromatography on a 4 g ISCO® cartridge eluting with a gradient of 100% dichloromethane to 10% methanol in dichloromethane to give the title compound (35 mg but obtained not fully pure); further purification attempts were unsuccessful.

¹H-NMR (400 MHz, DMSO-d₆) [symbol] 1.15-1.79 (m, 10H), 6.05 (d, 1H), 7.05 (s, 1H), 7.19 (d, 1H) 7.24-7.28 (m, 1H), 7.55 (d, 1H), 8.14 (s, 1H), 9.51 (s, 1H). LRMS m/z (APCI) 508 [M+H]⁺

### Example 22

### {2-[(8′-chloro-2′-oxo-2′,3′-dihydro-1′H-spiro[cyclohexane-1,4′-quinazolin]-5′-yl)oxy]-3-fluorophenyl}acetic acid

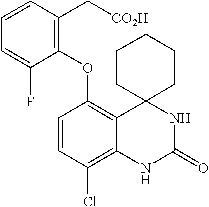

The product of Preparation 34 (50 mg, 0.13 mmol) was suspended in 1:1:1 acetic acid: H₂O: H₂SO₄ (2 ml) and heated to 90° C. for 18 hours. Water (10 ml) was added to afford a white precipitate, which was collected by filtration then dissolved in 2M NaOH solution (20 ml). This solution was washed with ethyl acetate (20 ml) and then re-acidified using 2M HCl to yield a white solid. The solid was collected by filtration and dried in vacuo to yield the title compound (23 mg, 0.055 mmol, 42%); LCMS showed the presence of a 5% impurity.

¹H-NMR (400 MHz, DMSO-d₆) δ 1.17 (m, 1H), 1.45 (m, 2H), 1.56 (m, 1H), 1.67-1.83 (m, 4H), 2.46 (m obscured by DMSO peak, assumed 2H), 3.44 (d, 1H), 3.61 (d, 1H), 6.03 (d, 1H), 7.10 (s, NH), 7.17 (d, 1H), 7.25 (m, 3H), 8.18 (s, NH).

LCMS m/z (ESI) 419 [M+H]⁺

### Example 23

### (a) Tert-butyl {2-[(8′-chloro-2′-oxo-2′,3′-dihydro-1′H-spiro[cyclohexane-1,4′-quinazolin]-5′-yl)oxy]phenoxy}acetate

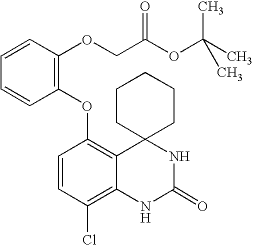

Tert-butyl bromoacetate (60 μl, 0.408 mmol) was added to the impure product of Preparation 35 (122 mg, 0.340 mmol) and Cs₂CO₃ (133 mg, 0.408 mmol) in DMF (2 ml) and the reaction heated at 90° C. for 4 hours then cooled overnight. The reaction was partitioned between ethyl acetate and water, the ethyl acetate extract washed with brine, dried over MgSO₄ and evaporated in vacuo to give ˜150 mg yellow oil. The residue was purified on a 4 g ISCO® column eluting with a gradient of 100% heptane to 100% ethyl acetate to give the title compound (68 mg, 0.143 mmol, 24%).

¹H-NMR (CDCl₃, 400 MHz) δ 1.27-1.37 (m, 2H), 1.45 (s, 9H), 1.56-1.60 (m, 2H), 1.69-1.75 (m, 2H), 1.92-1.95 (m, 2H), 2.65-2.73 (m, 2H), 5.92 (s, 1H), 6.30 (d, 1H), 6.91 (d, 1H), 7.00-7.03 (m, 2H), 7.08 (d, 1H), 7.14-7.18 (m, 2H).

LRMS m/z (APCI) 473 [M+H]⁺

### (b) {2-[(8′-chloro-2′-oxo-2′,3′-dihydro-1′H-spiro[cyclohexane-1,4′-quinazolin]-5′-yl)oxy]phenoxy}acetic acid

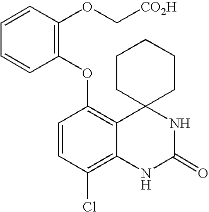

3:1 DCM:TFA (1.3 ml) was added to the product of step (a) and the mixture stirred under a nitrogen atmosphere for 7 hours before evaporating in vacuo (bath temperature at 60° C.). Purification on a 4 g ISCO® column eluting with a gradient of 100% heptane to 100% ethyl acetate afforded the title compound as a white solid (18 mg, 0.043 mmol, 54%).

¹H-NMR (CDCl₃, 400 MHz) δ 1.21-1.29 (m, 2H), 1.51-1.64 (m, 4H), 1.92 (d, 2H), 2.64-2.71 (m, 2H), 4.63 (s, 2H), 6.31 (d, 1H), 6.92 (s, 1H), 7.01-7.04 (m, 3H), 7.08 (d, 1H) 7.15-7.20 (m, 1H), 7.54 (s, 1H).

LRMS m/z (ESI) 417 [M+H]⁺

### Example 24

### (a) tert-butyl {4-[(8′-chloro-2′-oxo-2′,3′-dihydro-1′H-spiro[cyclohexane-1,4′-quinazolin]-5-yl)oxy]phenoxy}acetate

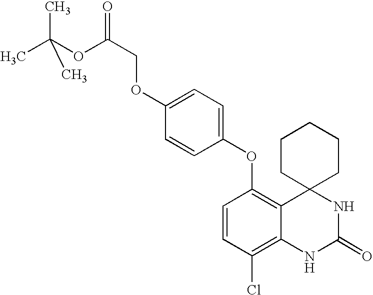

The title compound (25 mg, 0.534 mmol, 33%) was prepared by in a similar manner to Example 23 step (a) starting with the product of Preparation 36 (57 mg, 0.16 mmol).

¹H-NMR (CDCl₃, 400 MHz) δ 1.20-1.30 (m, 2H), 1.49 (s, 9H), 1.64-1.74 (m, 2H), 1.80-1.91 (m, 4H), 2.46-2.54 (m, 2H), 4.50 (s, 2H), 5.78 (s, 1H), 6.32 (d, 1H), 6.88-6.93 (m, 4H), 7.10 (broad s, 1H), 7.11 (d, 1H).

LRMS m/z (APCI) 473 [m+H]⁻

### (b) {4-[(8′-chloro-2′-oxo-2′,3′-dihydro-1′H-spiro[cyclohexane-1,4′-quinazolin]-5′-yl)oxy]phenoxy}acetic acid

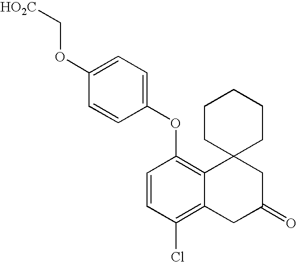

The title compound (10 mg, 0.024 mmol, 45%) was prepared in a similar manner to Example 23 starting with the product of step (a) (25 mg, 0.053 mmol).

¹H-NMR (400 MHz, CD₃OD) δ 1.22-1.28 (m, 2H), 1.59-1.66 (m, 2H), 1.72 (d, 2H), 1.86 (d, 2H), 2.50-2.58 (m, 2H), 4.66 (s, 2H), 6.37 (d, 1H), 6.94-7.00 (m, 4H), 7.19 (d, 1H).

LRMS m/z (ESI) 417 [M+H]⁺

### Example 25

### Methyl 2-[(8′-chloro-2′-oxo-2′,3′-dihydro-1′H-spiro[cyclohexane-1,4′-quinazolin]-5′-yl)oxy]-3-fluorobenzoate

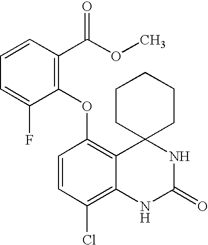

The product of Example 6 (1 g. 2.47 mmol) was suspended in methanol (50 ml) and H₂SO₄ (0.5 ml) was added dropwise. The reaction was then heated at reflux for 72 hours. The methanol had completely evaporated, leaving behind a white solid and the H₂SO₄. Water was added and the solid was filtered and dried in vacuo to afford the title compound as a white solid (920 mg, 2.19 mmol, 88.9%).

¹H-NMR (DMSO-d₆, 400 MHz) δ 1.12 (m, 1H), 1.44 (m, 2H), 1.60 (m, 1H), 1.71-1.88 (m, 4H), 2.56 (m, 2H), 3.70 (s, 3H), 6.00 (d, 1H), 7.10 (s, NH), 7.15 (d, 1H), 7.43 (m, 1H), 7.64 (t, 1H), 7.77 (d, 1H), 8.17 (s, NH).

LCMS m/z (ESI) 419 [M+H]⁺

### Preparation 1

### 8′-chloro-2′-oxo-2′,3′-dihydro-1′H-spiro[cyclohexane-1,4′-quinazolin]-5′-yl trifluoromethanesulfonate

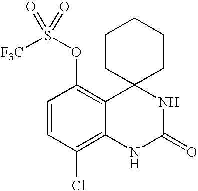

8′-Chloro-5′-hydroxy-1′H-spiro[cyclohexane-1,4′-quinazolin]-2′(3′H)-one (prepared as described in Bioorg. Med. Chem. Lett, (2004), 14 (18), 4627-4632) (5.0 g, 18.7 mmol) was dissolved in THF (70 ml) and sodium hydride (60% dispersion in oil, 0.790 g, 20.6 mmol) added in portions; very gentle effervescence was seen. Once addition was complete the suspension was warmed at 40° C. for 30 minutes then cooled to room temperature. A solution of N-phenyl-bis(trifluoromethanesulfonimide) (8.04 g, 22.5 mmol) in THF (30 ml) was added dropwise (exotherm observed). The reaction was stirred at room temperature for 2 hours, further N-phenyl-bis(trifluoromethanesulfonimide) (0.670 g, 1.87 mmol as a 5 ml solution in THF) was added and stirring continued for 18 hours. The mixture was diluted with ethyl acetate (150 ml) and washed with water (150 ml), 2M NaOH (2×150 ml) and saturated brine (100 ml) and dried over MgSO₄. On standing at room temperature the product precipitated. The product was collected by filtration and washed with ethyl acetate (50 ml) and diethyl ether (50 ml) to give 4.63 g white solid. The mother liquors were reduced in volume to around 100 ml to precipitate further product. Both crops were combined and dried in vacuo at 50° C. to give the title compound as a white solid (6.19 g, 15.5 mmol, 82%).

¹H-NMR (CDCl₃, 400 MHz): δ 1.31-2.27 (10H, m), 5.51 (1H, broad s), 6.93 (1H, d), 7.19 (1H, broad s), 7.33 (1H, d).

LRMS m/z (ESI) 399 [M+H]⁺

### Preparation 2

### 5-[(8′-chloro-2′-oxo-2′,3′-dihydro-1′H-spiro[cyclohexane-1,4′-quinazolin]-5′-yl)]-2-fluorobenzonitrile

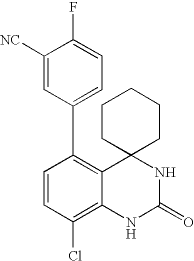

A mixture of the product of Preparation 1 (9.08 g, 22.8 mmol), 3-cyano-4-fluorophenylboronic acid (5.63 g, 34.2 mmol) and 2M aqueous sodium carbonate in toluene (140 ml) and methanol (20 ml) at room temperature was purged with argon for 1 hour. The mixture was heated to 100° C., palladium tetrakis(triphenylphosphine) (1.32 g, 1.14 mmol) was added under argon, the mixture stirred vigorously for 6 hours and allowed to cool. The mixture was poured into heptane (300 ml) and water (200 ml) and stirred for 30 minutes. The resulting solid was collected by filtration washing with water, heptane and finally diethyl ether (3×50 ml), which gave after air drying a brown solid (9.48 g). The solid was purified by column chromatography on silica eluting with dichloromethane: ethyl acetate (9:1) and recrystallised from acetic acid:water to give the title compound, as a 1:1 acetic acid solvate, as a pale brown solid (7.27 g, 19.65 mmol, 86%).

¹H-NMR (CDCl₃, 400 MHz): [symbol] 0.85 (m, 1H), 1.25 (m, 2H), 1.51 (m, 3H), 1.63 (m, 2H), 1.85 (m, 2H), 5.48 (s, 1H), 6.59 (d, 1H), 7.28 (d, 1H), 7.37 (s, 1H), 7.49 (m, 3H).

LRMS m/z (ESI) 370[M+H]⁺

### Preparation 3

### 3-[(8′-chloro-2′-oxo-2′,3′-dihydro-1′H-spiro[cyclohexane-1,4′-quinazolin]-5′-yl)]benzonitrile

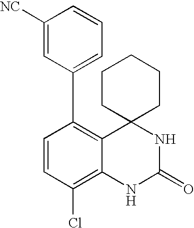

The title compound (27 mg, 0.076 mmol, 61%) was prepared in a similar manner to Preparation 2 starting with the product of Preparation 1 (50 mg, 0.13 mmol) and 3-cyanophenylboronic acid.

¹H-NMR (DMSO-d₆, 400 MHz) δ 0.64-1.64 (m, 10H), 6.60 (d, 1H), 6.83 (broad s, 1H), 7.34 (d, 1H), 7.61 (d, 2H), 7.78 (broad s, 1H), 7.87 (m, 1H), 8.45 (broad s, 1H)

LRMS m/z (ESI) 352[M+H]⁺

### Preparation 4

### 4-(8′-chloro-2′-methylene-2′,3′-dihydro-1′H-spiro[cyclohexane-1,4′-quinazolin]-5′-yl)-2-fluorobenzaldehyde

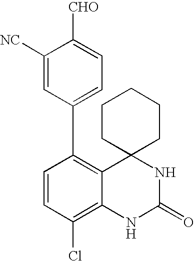

To a solution of the product of Preparation 1 (100 mg, 0.502 mmol), 3-fluoro-4-formylphenylboronic acid (126 mg, 0.376 mmol), Na₂CO₃ (0.376 ml of a 2M aqueous solution) in dimethoxyethane (1 ml) was added bis(triphenyl-phosphine)palladium (II) chloride (17.6 mg, 0.025 mmol) in a microwave tube. The tube was purged with nitrogen and then heated for 30 minutes at 120° C. in the microwave reactor. Further boronic acid (84 mg, 1 eq) and Pd(PPh₃)Cl₂ (17.6 mg, 0.05 eq) was added and the reaction returned to microwave for 15 minutes at 120° C. The solvent was removed in vacuo and the residue partitioned between 2M NaOH (15 ml) and ethyl acetate (15 ml) and further washed with 2M NaOH (15 ml). The organics were then dried over MgSO₄, purified on a 12 g ISCO® column eluting 0-40% ethyl acetate in heptane to afford the title compound as a white solid (15 mg, 0.0402 mmol, 8%).

¹H-NMR (400 MHz, CDCl₃) δ 1.24-1.63 (8H, m), 1.85 (2H, t), 6.62 (1H, d), 7.08 (1H, d), 7.16 (1H, d), 7.28 (1H, m), 7.90 (1H, t), 10.42 (1H, s).

LRMS m/z (ESI) 373[M+H]⁺

### Preparation 5

### 8′-chloro-5′-(3-hydroxyphenyl)-1′H-spiro[cyclohexane-1,4′-quinazolin]-2′(3′H)-one

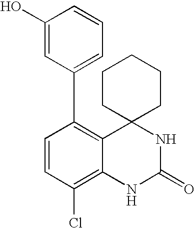

The title compound (274 mg, 0.8 mmol, 64%) was prepared in a similar manner to Preparation 2 (starting with the product of Preparation 1 (500 mg, 1.254 mmol) and 3-hydroxyphenylboronic acid.

¹H-NMR (DMSO-d₆, 400 MHz) δ 0.71 (m, 1H), 1.25 (m, 2H), 1.41 (m, 3H), 1.59 (m, 4H), 6.62 (m, 3H), 6.75 (m, 2H), 7.16 (m, 1H), 7.28 (d, 1H), 8.30 (s, 1H), 9.53 (s, 1H).

LRMS m/z (ESI) 343[M+H]⁺

### Preparation 6

### 2-{(8′-chloro-2′-oxo-2′,3′-dihydro-1′H-spiro[cyclohexane-1,4′-quinazolin]-5′-yl)oxy}-3-fluorobenzonitrile

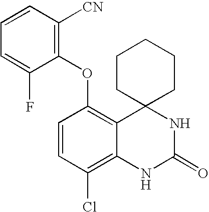

To a partial solution of 8′-chloro-5′-hydroxy-1H-spiro[cyclohexane-1,4′-quinazolin]-2′(3′H)-one (prepared as described in Bioorg. Med. Chem. Lett, (2004), 14 (18), 4627-4632) (30.0 g, 112 mmol) in DMF (250 ml) was added cesium carbonate (55.0 g, 169 mmol) and 2,3-difluorobenzonitrile (12.5 g, 135 mmol) as a solution in DMF (50 ml) in one portion and the mixture was heated at 80° C. for 20 hours. The reaction was cooled, water (400 ml) was added and the suspension was stirred for 2 hours. The resulting solid was collected by filtration, washing with water (100 ml), air-dried then stirred with water (100 ml), filtered and air-dried—this was repeated until the filtrate was colourless (3 times in total). The off-white solid was dried in vacuo at 50° C. to give the title compound (42.9 g, 111 mmol, 99%).

¹H-NMR (DMSO-d₆, 400 MHz) δ 1.15 (m, 1H), 1.49 (m, 2H), 1.61 (m, 1H), 1.81 (m, 4H), 2.43 (m, 2H), 6.20 (d, 1H), 7.17 (s, 1H), 7.23 (d, 1H), 7.51 (m, 1H), 7.83 (d, 2H), 8.33 (s, 1H).

LRMS m/z 386 [M+H]⁺

### Preparation 7

### (a) 8′-Chloro-5′-methoxy-1′H-spiro[cyclopentyl-1,4′-quinazoline]-2′(3′H)-one

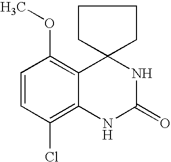

To 2-chloro-5-methoxyphenylurea (WO 02/074754, intermediate 5) (22.04 g, 0.11 mol) was added Eaton's reagent (a 7.7 wt. % solution of phosphorus (V) oxide in methanesulphonic acid) (440.8 ml) followed by cyclopentanone (19.5 ml, 0.22 mol) and the resulting solution heated at 85° C. for 4 hours. The reaction was cooled to −5° C. and water added cautiously keeping the temperature between 20 and 30° C. Dichloromethane (400 ml in total) and brine (200 ml) were then added and the phases separated. The aqueous phase was washed with dichloromethane (2×100 ml), the organic extracts combined and evaporated in vacuo to give a dark oil which was purified on a silica chromatography column eluting with dichloromethane:methanol (95:5 to 90:10) to give the product as a dark brown solid. The solid was triturated with diethyl ether and pentane, collected by filtration and dried to give the title compound as a brown solid (27.17 g, 0.1 mol, 92%).

¹H-NMR (400 MHz, CDCl₃): δ 1.7-1.8 (m, 6H), 2.4-2.5 (m, 2H), 3.7 (s, 3H), 5.75 (br s, 1H), 6.4 (d, 1H), 7.05 (s, 1H), 7.15 (d, 1H).

LRMS m/z (APCI) 267[M+H]⁺

### (b) 8′-chloro-5′-hydroxy-1′H-spiro[cyclopentane-1,4′-quinazolin]-2′(3′H)-one

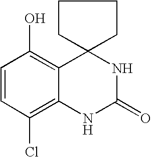

To the compound of step (a) (25 g, 0.093 mol) was added acetic acid (250 ml) followed by 48% aqueous hydrobromic acid (207 ml, 1.86 mol) in one portion and the resulting solution stirred at 115° C. for 7 days. The reaction mixture was cooled to 100° C. and water (207 ml) was added dropwise. The mixture was concentrated in vacuo to precipitate a brown solid which was collected by filtration and washed with water (2×100 ml). A second portion of product was obtained from the filtrate on standing. The combined portions of product were dried by slurry with toluene (150 ml) and solvent removal in vacuo three times to give a grey solid which was pre-absorbed onto silica and purified by column chromatography eluting with dichloromethane:methanol (98:2 to 95:5 to 80:20). The product fractions were concentrated in vacuo and the resulting solid triturated with pentane and filtered to afford the title compound as a brown solid (10 g, 0.0395 mol, 42%).

¹H-NMR (400 MHz, DMSO-d₆) δ 1.6-1.8 (m, 6H), 2.3-2.4 (m, 2H), 6.4 (d, 1H), 7.11 (d, 1H), 7.2 (s, 1H), 7.8 (s, 1H), 9.9 (s, 1H).

LRMS m/z (ESI) 253[M+H]⁺

### Preparation 8

### 2-{(8′-chloro-2′oxo-2′,3′-dihydro-1′H-spiro[cyclohexane-1,4′-quinazolin]-5′-yl)oxy}-3-fluorobenzonitrile

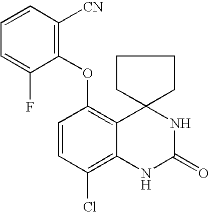

The title compound (27.2 g, 73 mmol, 92%) was prepared in a similar manner to Preparation 6 starting with the product of Preparation 7 (20 g, 79 mmol) and 2,3-difluorobenzonitrile.

¹H-NMR (DMSOd-₆, 400 MHz) δ 1.63 (m, 2H), 1.84 (m, 4H), 2.36 (m, 2H) 6.19 (d, 1H), 7.21 (d, 1H), 7.48 (s, 1H), 7.52 (m, 1H), 7.82 (m, 2H), 8.36 (s, 1H). LRMS m/z (ESI) 372[M+H]⁺

### Preparation 9

### 3-chloro-2-[(8′-chloro-2′-oxo-2′,3′-dihydro-1′H-spiro[cyclohexane-1,4′-quinazolin]-5′-yl)oxy]benzonitrile

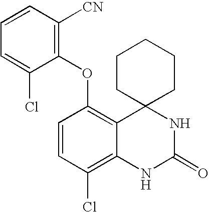

The title compound (550 mg, 1.37 mmol, 73%) was prepared in a similar manner to Preparation 6 starting with 8′-chloro-5′-hydroxy-1′H-spiro[cyclohexane-1,4′-quinazolin]-2′(3′H)-one (prepared as described in Bioorg. Med. Chem. Lett, (2004), 14 (18), 4627-4632) (500 mg, 1.87 mmol) and 3-chloro-2-fluorobenzonitrile.

¹H-NMR (DMSO-d₆, 400 MHz) δ 1.19 (m, 1H), 1.48 (m, 2H), 1.61 (m, 1H), 1.82 (m, 4H), 2.77 (m, 2H), 5.99 (d, 1H), 7.13 (broad s, 1H), 7.20 (d, 1H), 7.55 (t, 1H), 8.00 (m, 2H), 8.16 (broad s, 1H).

LCMS (ESI) 403 [M+H]⁺

### Preparation 10

### 3-chloro-2-[(8′-fluoro-2′-oxo-2′,3′-dihydro-1′H-spiro[cyclohexane-1,4′-quinazolin]-5′-yl)oxy]benzonitrile

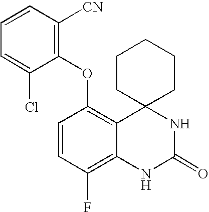

The title compound (14.1 g, 36.5 mmol, 97%) was prepared in a similar manner to Preparation 7 starting with 8′-fluoro-5′-hydroxy-1′H-spiro[cyclohexane-1,4′-quinazolin]-2′(3′H)-one (described in WO 2004/026818, intermediate c) (10 g, 37 mmol) and 3-chloro-2-fluorobenzonitrile.

¹HNMR (DMSO-d₆, 400 MHz) δ 1.13 (m, 1H), 1.48 (m, 2H), 1.61 (m, 1H), 1.75-1.86 (m, 4H), 2.53 (m, 2H), 5.87 (m, 1H), 6.93 (broad s, 1H), 6.97 (t, 1H), 7.51 (t, 1H), 7.98 (m, 2H), 9.14 (broad s, 1H).

LRMS m/z (ESI) 386 [M+H]⁺

### Preparation 11

### 5-fluoro-2-[(8′-fluoro-2′-oxo-2′,3′-dihydro-1′H-spiro[cyclohexane-1,4′-quinazolin]-5′-yl)oxy]benzonitrile

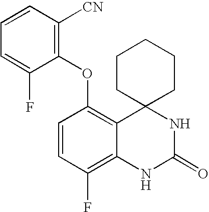

The title compound (1.38 g, 3.73 mmol, 92%) was prepared in a similar manner to the compound of Preparation 10 starting with 8′-fluoro-5′-hydroxy-1′H-spiro-[cyclohexane-1,4′-quinazolin]-2′(3′H)-one (described in WO 2004/026818, intermediate c) (1.0 g, 4.0 mmol) and 2,5-difluorobenzonitrile.

¹H-NMR (DMSO-d₆, 400 MHz) δ 1.12 (m, 1H), 1.44 (m, 2H), 1.60 (m, 1H), 1.80 (m, 4H), 2.43 (m, 2H), 6.18 (m, 1H), 7.00 (m, 2H), 5.50 (m, 1H), 7.79 (m, 2H), 9.19 (s, 1H).

LRMS m/z (APCI) 370 [M+H]⁺

### Preparation 12

### 5-fluoro-2-[(8′-chloro-2′-oxo-2′,3′-dihydro-1′H-spiro[cyclohexane-1,4′-quinazolin]-5′-yl)oxy]benzonitrile

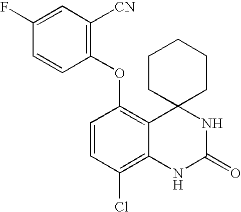

The title compound (10.53 g, 27 mmol, 91%) was prepared in a similar manner to the compound of Preparation 6 starting with 8′-chloro-5′-hydroxy-1′H-spiro[cyclohexane-1,4′-quinazolin]-2′(3′H)-one (prepared as described in Bioorg. Med. Chem. Lett, (2004), 14 (18), 4627-4632) (8.0 g, 30.0 mmol) and 2,5-difluorobenzonitrile.

¹H-NMR (DMSO-d₆, 400 MHz) δ 1.10 (m, 1H), 1.43 (m, 2H), 1.60 (m, 1H), 1.77 (m, 4H), 2.20 (m, 2H), 6.53 (d, 1H), 7.10 (m, 1H), 7.13 (s, 1H), 7.34 (d, 1H), 7.56 (m, 1H), 7.93 (m, 1H), 8.36 (s, 1H).

LRMS m/z (ESI) 386[M+H]⁺

### Preparation 13

### 5-fluoro-2-[(8′-chloro-2′-oxo-2′,3′-dihydro-1′H-spiro[pentane-1,4′-quinazolin]-5′-yl)oxy]benzonitrile

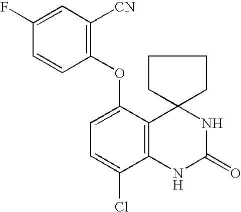

The title compound (4.4 g, 11.8 mmol, 60%) was prepared in a similar manner to the compound of Preparation 8 starting with the compound of Preparation 7 (5.0 g, 19.6 mmol) and 2,5-difluorobenzonitrile.

¹H-NMR (DMSO-d₆, 400 MHz) δ 1.60 (m, 2H), 1.81 (m, 4H), 2.20 (m, 2H), 6.46 (d, 1H), 7.21 (m, 1H), 7.30 (d, 1H), 7.44 (s, 1H), 7.58 (m, 1H), 7.92 (m, 1H), 8.37 (s, 1H).

LRMS m/z (APCI) 372[M+H]⁺

### Preparation 14

### 3-fluoro-2-[(8′-chloro-2′-oxo-2′,3′-dihydro-1′H-spiro[pentane-1,4′-quinazolin]-5′-yl)oxy]benzonitrile

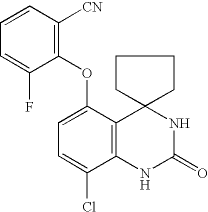

The title compound (27.2 g, 73.7 mmol, 92%) was prepared in a similar manner to the compound of Preparation 8 starting with the compound of Preparation 7 (20.0 g, 79.1 mmol) and 2,3-difluorobenzonitrile.

¹H-NMR (DMSO-d₆, 400 MHz) δ 1.65 (broad s, 2H), 1.85 (broad s, 5H), 3.30 (broad s, 1H), 6.21 (d, 1H), 7.23 (d, 1H), 7.48 (s, 1H), 7.49-7.55 (m, 1H), 7.79-7.84 (m, 2H) 8.33 (s, 1H).

LRMS m/z (APCI) 372 [M+H]⁺

### Preparation 15

### 3-chloro-2-[(8′-chloro-2′-oxo-2′,3′-dihydro-1′H-spiro[pentane-1,4′-quinazolin]-5′-yl)oxy]benzonitrile

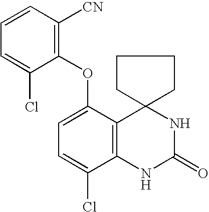

The title compound (6.19 g, 15.9 mmol, 99%) was prepared in a similar manner to the compound of Preparation 8 starting with the compound of Preparation 7 (4.2 g, 16.01 mmol) and 2-fluoro-3-chlorobenzonitrile.

¹H-NMR (DMSO-d₆, 400 MHz) δ 1.68 (broad s, 2H), 1.80 (m, 4H), 2.57 (m, 2H), 5.97 (d, 1H), 7.19 (d, 1H), 7.43 (s, 1H), 7.50 (m, 1H), 8.00 (m, 2H), 8.11 (s, 1H).

LRMS m/z (ESI) 388 [M+H]⁺

### Preparation 16

### 2-[(8′-chloro-2′-oxo-2′,3′-dihydro-1′H-spiro[cyclopentane-1,4′-quinazolin]-5′-yl)oxy]benzonitrile

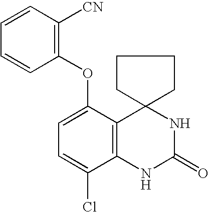

The title compound (6.74 g, 19.1 mmol, 100%) was prepared in a similar manner to the compound of Preparation 8 starting with the compound of Preparation 7 (5.0 g, 19.1 mmol) and 2-fluorobenzonitrile.

¹H-NMR (DMSO-d₆, 400 MHz) δ 1.58-1.66 (m, 2H), 1.78-1.87 (m, 4H), 2.17-2.26 (m, 2H), 6.51 (d, 1H), 7.14 (d, 1H), 7.30-7.35 (m, 2H), 7.45 (s, 1H), 7.67-7.72 (m, 1H), 7.91-7.93 (m, 1H), 8.38 (s, 1H).

LRMS m/z (APCI) 354 [MH]⁺

### Preparation 17

### 2-[(8′-chloro-2′-oxo-2′,3′-dihydro-1′H-spiro[cyclohexane-1,4′-quinazolin]-5′-yl)oxy]benzonitrile

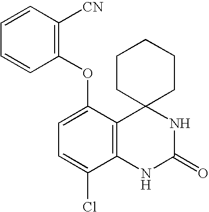

The title compound (99.48 g, 270 mmol, 96%) was prepared in a similar manner to the compound of Preparation 6 starting with 8′-chloro-5′-hydroxy-1′H-spiro[cyclohexane-1,4′-quinazolin]-2′(3′H)-one (prepared as described in Bioorg. Med. Chem. Lett, (2004), 14 (18), 4627-4632) (75.0 g, 281 mmol) and 2-fluorobenzonitrile.

¹H-NMR (CDCl₃, 400 MHz) δ 1.24-1.35 (m, 1H), 1.47-1.76 (m, 5H), 1.91-1.95 (m, 2H), 2.31-2.40 (m, 2H), 5.59 (s, 1H), 6.46 (d, 1H), 6.95 (d, 1H), 7.14 (s, 1H), 7.21-7.26 (m, 2H), 7.53-7.58 (m, 1H), 7.71-7.73 (m, 1H).

LRMS m/z (ESI) 368 [M+H]⁺

### Preparation 18

### 8′-chloro-5′-(2-fluoro-6-nitrophenoxy)-1′H-spiro[cyclohexane-1,4′-quinazolin]-2′(3′H)-one

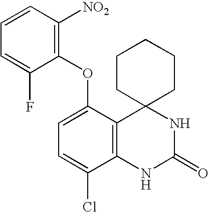

The title compound (346 mg, 0.853 mmol, 68%) was prepared in a similar manner to the compound of Preparation 6 starting with 8′-chloro-5′-hydroxy-1H-spiro[cyclohexane-1,4′-quinazolin]-2′(3′H)-one (prepared as described in Bioorg. Med. Chem. Lett, (2004) (200 mg, 1.26 mmol) and 2,3-difluoronitrobenzene.

¹H-NMR (DMSO-d₆, 400 MHz) δ1.11-1.20 (m, 1H), 1.44-1.51 (m, 2H), 1.57-1.62 (m, 1H), 1.73-1.85 (m, 4H), 2.33-2.39 (m, 2H), 6.24 (d, 1H), 7.12 (s, 1H), 7.21 (d, 1H), 7.55-7.63 (m, 1H), 7.83-7.88 (m, 1H), 8.02-8.04 (m, 1H), 8.26 (s, 1H).

LRMS m/z (APCI) 406 [M+H]⁺

### Preparation 19

### 5′-(2-amino-6-fluorophenoxy)-8′-chloro-1′H-spiro[cyclohexane-1,4′-quinazolin]-2′(3′H)-one

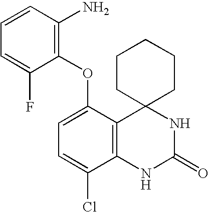

The compound of Preparation 18 (125 mg, 0.308 mmol) in acetic acid (4 ml) was hydrogenated over sulphided platinum on carbon (6 mg, 0.0308 mmol) under 1 atm of hydrogen at 50° C. The catalyst was removed by filtration, washing with acetic acid. Addition of water resulted in a suspension which was basified with NaOH pellets. The resulting slightly pink solid was collected by filtration and dried in vacuo to provide the title compound (78 mg, 0.207 mmol, 67%).

¹H-NMR (400 MHz, DMSO-d₆) δ 1.10-1.21 (m, 1H), 1.45-1.52 (m, 2H), 1.59-1.65 (m, 1H), 1.71-1.87 (m, 4H), 2.54-2.62 (m, 2H), 5.22 (s, 2H), 6.09 (d, 1H), 6.43-6.47 (m, 1H), 6.65 (d, 1H) 6.92-6.97 (m, 1H), 7.05 (s, 1H), 7.19 (d, 1H), 8.11 (s, 1H).

LRMS m/z (ESI) 376 [M+H]⁺

### Preparation 20

### 2-[(8′-chloro-2′-oxo-2′,3′-dihydro-1′H-spiro[cyclohexane-1,4′-quinazolin]-5′-yl)oxy]benzaldehyde

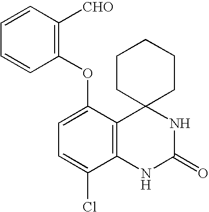

The title compound (180 mg, 0.485 mmol, 65%) was prepared in a similar manner to the compound of Preparation 6 starting with 8′-chloro-5′-hydroxy-1′H-spiro[cyclohexane-1,4′-quinazolin]-2′(3′H)-one (prepared as described in Bioorg. Med. Chem. Lett, (2004) (200 mg, 0.75 mmol) and 2-fluorobenzaldehyde.

¹H-NMR (CDCl₃, 400 MHz) δ 152-1.66 (m, 2H), 1.70-1.80 (m, 4H), 1.97-2.06 (m, 2H), 2.38-2.40 (m, 2H), 5.69 (s, 1H), 6.46 (d, 1H), 6.97 (d, 1H), 7.21 (s, 1H), 7.26 (d, 1H), 7.31-7.35 (m, 1H), 7.62-7.66 (m, 1H), 8.02-8.04 (m, 1H), 10.51 (s, 1H).

LRMS m/z (APCI) 371 [M+H]⁺.

### Preparation 21

### 4-[(8′-chloro-2′-oxo-2′,3′-dihydro-1′H-spiro[cyclohexane-1,4′-quinazolin]-5-yl)oxy]benzaldehyde

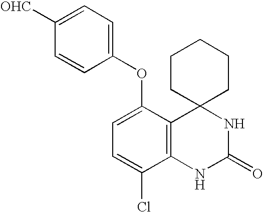

The title compound (750 mg, 2.02 mmol, 67%) was prepared in a similar manner to the compound of Preparation 6 starting with 8′-chloro-5′-hydroxy-1H-spiro[cyclohexane-1,4′-quinazolin]-2′(3′H)-one (prepared as described in Bioorg. Med. Chem. Lett, (2004) (800.0 mg, 3.0 mmol) and 4-fluoro-benzaldehyde.

¹H-NMR (DMSO-d₆, 400 MHz) δ 0.99 (m, 1H), 1.39 (m, 2H), 1.57 (m, 1H), 2.75 (m, 4H), 2.13 (m, 2H), 6.53 (d, 1H), 7.07 (s, NH), 7.18 (m, 2H), 7.36 (d, 1H), 7.92 (d, 1H), 8.35 (s, NH), 9.93 (s, 1H).

LCMS m/z (ESI) 371 [M+H]⁺

### Preparation 22

### 2-{(8′-chloro-2′-oxo-2′,3′-dihydro-1′H-spiro[cyclohexane-1,4′-quinazolin]-5′-yl)oxy}-3-fluorobenzecarboximidohydrazide

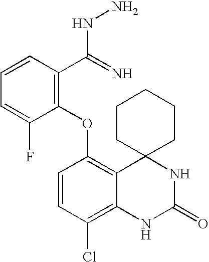

To a solution of the product of Preparation 6 (23 g, 60 mmol) in DMF (230 ml) was added hydrazine hydrate (5.78 mL, 1119 mmol) followed by phosphorus pentasulphide (660 mg, 2.98 mmol) and the reaction heated to 70° C. for 6 hours. A further portion of hydrazine hydrate (2.89 ml, 60 mmol) was added and the reaction continued for 18 hours. The reaction was cooled to room temperature and slowly poured into water (500 ml) with vigorous stirring. The resulting solid was collected by filtration. Solid was broken up and stirred in water to remove excess DMF, filtered and air-dried to yield a pale yellow solid (19.3 g, 46 mmol, 77%), which was used without further treatment.

¹H-NMR (DMSO-d₆, 400 MHz) δ 1.18-1.24, 1.43-1.59 and 1.65-1.83 (3×m, 8H), 2.56-2.64 (m, 2H), 4.81 (s, 2H), 5.46 (s, 2H), 5.99-6.02 (m, 1H), 7.04 (s, 1H), 7.12 (d, 1H), 7.27-7.37 (m, 3H), 8.10 (s, 1H).

LRMS m/z (APCI) 418[M+H]⁺

### Preparation 23

### 2-[(8′-chloro-2′-oxo-2′,3′-dihydro-1′H-spiro[cyclohexane-1,4′-quinazolin]-5′-yl)oxy]-5-fluorobenzenecarboximidohydrazide

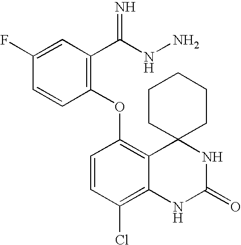

To a solution of the product of Preparation 12 (5.80 g, 15.0 mmol) in DMF (10 ml) at room temperature was added hydrazine hydrate (3.66 ml, 75 mmol) followed by P₂S₅ (167 mg, 0.75 mmol) and the resulting green reaction mixture heated to 70° C. for 5 hours, then allowed to cool to room temperature overnight. Water (60 ml) was added dropwise and the white emulsion stirred for 1 hour. The cream suspension was then poured into water (300 ml) and stirred for a further 1 hour. The resulting solid was collected by filtration, washed well with water and dried in vacuo to give the title compound as a white solid (6.01 g, 14.3 mmol, 95%).

¹H-NMR (DMSO-d₆, 400 MHz,) δ 1.16 (m, 2H), 1.42 (m, 2H), 1.53 (m, 1H), 1.68-1.85 (m, 3H), 2.39 (m, 2H), 4.91 (broad s, 2H), 5.47 (s, 2H), 6.24 (d, 1H), 6.91 (m, 1H), 7.04 (s, 1H), 7.14 (m, 1H), 7.21 (d, 1H), 7.29 (m, 1H), 8.17 (broad s, 1H).

LCMS (ESI) 418 [M+H]⁺

### Preparation 24

### 2-[(8′-chloro-2′-oxo-2′,3′-dihydro-1′H-spiro[cyclohexane-1,4′-quinazolin]-5′-yl)oxy]-3-fluorobenzenecarboximidohydrazide

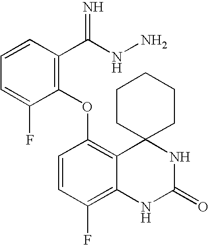

The title compound (150 mg, 0.37 mmol, 55%) was prepared in a similar manner to the compound of Preparation 23 starting with the product of Preparation 11 (250 mg, 0.677 mmol).

LRMS m/z (ESI) 402 [M+H]⁺, (APCI) 402 [M+H]⁺

### Preparation 25

### 2-[(8′-chloro-2′-oxo-2′,3′-dihydro-1′H-spiro[cyclopentane-1,4′-quinazolin]-5′-yl)oxy]-5-fluorobenzenecarboximidohydrazide

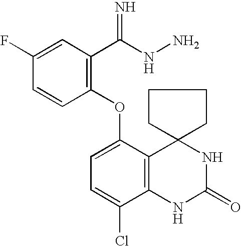

The title compound (300 mg, 0.74 mmol, 69%) was prepared in a similar manner to the compound of Preparation 23 starting with the product of Preparation 13 (396 mg, 1.07 mmol).

¹H-NMR (DMSO-d₆, 400 MHz) δ1.57-1.80 (m, 6H), 2.22-2.45 (m, 2H), 4.82 (s, 2H), 5.39 (s, 2H), 6.19 (d, 1H), 6.97 (m, 1H), 7.18 (m, 2H), 7.27 (m, 1H), 7.35 (s, NH), 8.17 (s, NH).

LCMS m/z (ESI) 404 [M+H]⁺

### Preparation 26

### 2-[(8′-chloro-2′-oxo-2′,3′-dihydro-1′H-spiro[cyclopentane-1,4′-quinazolin]-5′-yl)oxy]-3-fluorobenzenecarboximidohydrazide

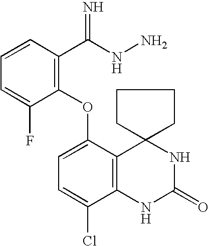

The title compound (1.6 g, 3.9 mmol, 33%) was prepared in a similar manner to the compound of Preparation 23 starting with the product of Preparation 14 (4.4 g, 12.0 mmol).

¹H-NMR (DMSO-d₆, 400 MHz) δ 1.66-1.82 (m, 7H), 2.60 (broad s, 1H), 4.81 (broad s, 2H), 5.39 (broad s, 2H), 6.01 (d, 1H), 7.12 (d, 1H), 7.29-7.37 (m, 4H), 8.07 (s, 1H).

LRMS m/z (APCI) 404 [M+H]⁺

### Preparation 27

### 2-[(8′-chloro-2′-oxo-2′,3′-dihydro-1′H-spiro[cyclopentane-1,4′-quinazolin]-5′-yl)oxy]-3-chlorobenzenecarboximidohydrazide

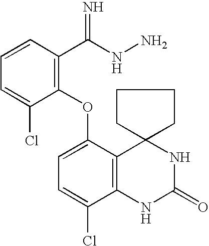

The title compound (7.0 g, crude, quantitative) was prepared in a similar manner to the compound of Preparation 23 starting with the product of Preparation 15 (6.19 g, 15.94 mmol).

¹H-NMR (DMSO-d₆, 400 MHz) δ 1.63-1.82 (m, 6H), 2.67 (m, 2H), 4.72 (broad s, 2H), 5.38 (broad s, 2H), 5.82 (d, 1H), 7.07 (d, 1H), 7.32 (m, 2H), 7.42 (m, 1H), 7.57 (m, 1H), 8.05 (broad s, 1H).

LRMS m/z (ESI) 420 [M+H]⁺

### Preparation 28

### 2-[(8′-chloro-2′-oxo-2′,3′-dihydro-1′H-spiro[cyclopentane-1,4′-quinazolin]-5′-yl)oxy]benzamide

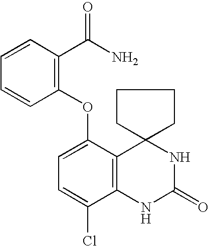

Concentrated H₂SO₄ (90 ml) was added to the product of Preparation 16 (6.77 g, 19.1 mmol) suspended in water (30 ml) and the resulting brown solution was heated to 100° C. for approximately 1 hour, cooled to room temperature and poured into water (200 ml). The resulting solid was collected by filtration, washing firstly with 2N NaOH (200 ml) and then with water, air dried and then in vacuo at 70° C. to afford the title compound as a beige solid (6.04 g, 16.2 mmol, 85%).

¹H-NMR (DMSO-d₆, 400 MHz) δ 1.57-1.63 (m, 2H), 1.71-1.82 (m, 4H), 2.25-2.34 (m, 2H), 6.33 (d, 1H), 6.94 (d, 1H), 7.19-7.24 (m, 2H), 7.35 (s, 1H), 7.40-7.45 (m, 2H), 7.56 (broad s, 1H) 7.58-7.61 (m, 1H), 8.20 (broad s, 1H).

LRMS m/z (APCI) 372 [M+H]⁺ (ESI) 372 [M+H]⁺

### Preparation 29

### 2-[(8′-chloro-2′-oxo-2′,3′-dihydro-1′H-spiro[cyclopentane-1,4′-quinazolin]-5′-yl)oxy]benzenecarboximidohydrazide

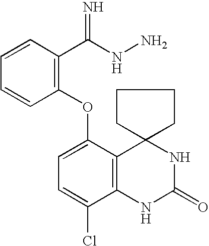

### a) Methyl 2-[(8′-chloro-2′-oxo-2′,3′-dihydro-1′H-spiro[cyclohexane-1,4′-quinazolin]-5′-yl)oxy]benzenecarboximidoate

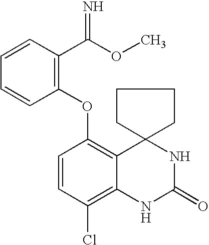

Trimethyloxonium tetrafluoroborate (2.64 g, 17.9 mmol) was added to the product of Preparation 28 (6.04 g, 16.2 mmol) in DCM (300 ml) at room temperature and the resulting suspension stirred under a nitrogen atmosphere for 18 hours. Methanol (3 ml) was added to quench the excess trimethyloxonium tetrafluoroborate and stirred for 10 minutes. The product was used directly in step b).

### b) 2-[(8′-chloro-2′-oxo-2′,3′-dihydro-1′H-spiro[cyclopentane-1,4′-quinazolin]-5′-yl)oxy]benzenecarboximidohydrazide

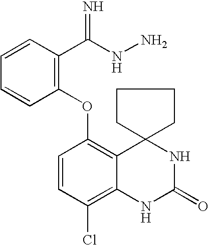

Hydrazine hydrate (2.4 ml, 48.7 mmol) was added to the solution obtained in step a) and the mixture heated at 50° C. After 4 hours the reaction was evaporated to dryness in vacuo. 2N HCl was added, then the mixture washed with ethyl acetate. The aqueous HCl was basified with NaOH pellets (exothermic) and the product extracted into ethyl acetate. The organic extract was backwashed with brine, dried over MgSO₄ and concentrated to give the title compound (2.18 g, 5.65 mmol, 35%) which was used without further purification.

LRMS (APCI) 386 [M+H]⁺ (ESI) 386 [M+H]⁺

### Preparation 30

### 2-[(8′-chloro-2′-oxo-2′,3′-dihydro-1′H-spiro[cyclopentane-1,4′-quinazolin]-5′-yl)oxy]benzamide

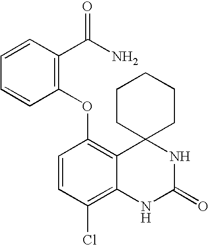

The title compound (11.3 g, crude, quantitative) was prepared in similar manner to the compound of Preparation 28 starting from the product of Preparation 17 (10.4 g, 28.3 mmol).

¹H-NMR (DMSO-d₆, 400 MHz) δ 1.09-1.19 (m, 1H), 1.41-1.43 (m, 2H), 1.54-1.58 (m, 1H), 1.70-1.84 (m, 4H), 2.30-2.37 (m, 2H), 6.38 (d, 1H), 6.88 (d, 1H), 7.05 (s, 1H), 7.19-7.23 (m, 1H), 7.28 (d, 1H) 7.40-7.44 (m, 1H), 7.49 (s, 1H), 7.60-7.62 (m, 2H), 8.22 (s, 1H).

LRMS (ESI) 386 [M+H]⁺

### Preparation 31

### 2-[(8′-chloro-2′-oxo-2′,3′-dihydro-1′H-spiro[cyclopentane-1,4′-quinazolin]-5′-yl)oxy]benzenecarboximidohydrazide

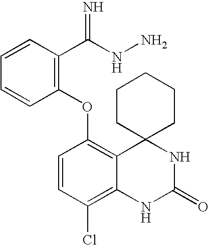

### a) Methyl 2-[(8′-chloro-2′-oxo-2′,3′-dihydro-1′H-spiro[cyclohexane-1,4′-quinazolin]-5′-yl)oxy]benzenecarboximidoate

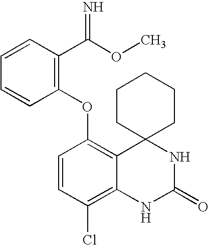

The product of Preparation 30 (6.15 g, 15.9 mmol) was suspended in DCM (250 ml) and trimethyloxonium tetrafluoroborate (2.48 g, 16.7 mmol) was then added in one portion and the reaction stirred at room temperature. After 4 hours methanol was added to quench the trimethyloxonium tetrafluoroborate. The product was used directly in step b).

LRMS m/z (APCI) 400 [M+H]⁺, (ESI) 400 [M+H]⁺

### b) 2-[(8′-chloro-2′-oxo-2′,3′-dihydro-1′H-spiro[cyclopentane-1,4′-quinazolin]-5′-yl)oxy]benzenecarboximidohydrazide

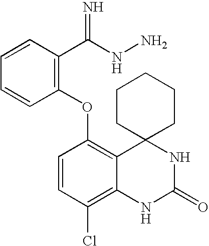

Hydrazine hydrate (2.32 ml, 47.8 mmol) was added to the suspension from step a) and the resulting clear orange-yellow solution stirred at room temperature for 18 hours. 2N HCl was added to the reaction mixture which caused a large quantity of material to separate as a gum. The solution was decanted and back washed with HCl to extract any remaining product. The gum was then dissolved with methanol/ethyl acetate and added to the HCl washings, adding more 2N HCl to ensure pH 1. The phases were separated and the acidic aqueous phase basified to pH>12 with NaOH pellets and causing a blue colouration. The product was extracted into ethyl acetate, washed with brine, dried over MgSO₄ and evaporated in vacuo to give the title compound as a faintly blue coloured solid (3.77 g, 9.42 mmol, 59%) which was used without further purification.

¹H-NMR (DMSO-d₆, 400 MHz) δ 1.09-1.19 (m, 1H), 1.41-1.43 (m, 2H), 1.54-1.58 (m, 1H), 1.70-1.84 (m, 4H), 2.30-2.37 (m, 2H), 6.38 (d, 1H), 6.88 (d, 1H), 7.05 (s, 1H), 7.19-7.23 (m, 1H), 7.28 (d, 1H) 7.40-7.44 (m, 1H), 7.49 (s, 1H), 7.60-7.62 (m, 2H), 8.22 (s, 1H).

LRMS m/z (ESI) 386 [M+H]+

### Preparation 32

### 8′-chloro-5′-[2-fluoro-6-(hydroxymethyl)phenoxy]-1′H-spiro[cyclohexane-1,4′-quinazolin]-2′(3′H)-one

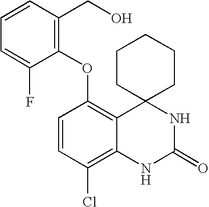

The product of Example 25 (520 mg, 1.24 mmol) was dissolved in THF (10 ml), to which was added 2M LiBH₄ in THF (1.24 ml, 2.48 mmol) dropwise. The reaction was then heated to 80° C. before adding 2 drops of methanol. The reaction was then left to reflux for 18 hours. A white precipitate had formed, which was caked to the bottom of the flask. Saturated aqueous sodium bicarbonate was added (20 ml) and the product extracted into ethyl acetate (20 ml). The organics were dried over Na₂SO₄ and concentrated in vacuo to afford the title compound as a white solid (370 mg, 0.95 mmol, 76.3%).

¹H-NMR (DMSO-d₆, 400 MHz) δ 1.12 (m, 1H), 1.44 (m, 2H), 1.59 (m, 1H), 1.68-1.84 (m, 4H), 2.54 (m obscured by DMSO peak, assumed 2H), 4.38 (dd, 1H), 4.51 (dd, 1H), 5.27 (t, 1H), 6.00 (d, 1H), 7.11 (s, NH), 7.18 (d, 1H), 7.30 (m, 1H), 7.39 (d, 1H), 8.18 (s, NH).

LCMS m/z (ESI) 391 [M+H]⁺

### Preparation 33

### 8′-chloro-5′-[2-(chloromethyl)-6-fluorophenoxy]4H-spiro[cyclohexane-1,4′-quinazolin]-2′(3′H)-one

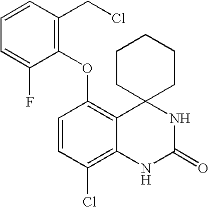

The product of Preparation 32 (370 mg, 0.5 mmol) and pyridine (82 mg, 1.4 mmol) were dissolved in DCM (10 ml) and cooled to 0° C. before adding thionyl chloride (146 mg, 1.23 mmol). The reaction was heated to reflux for 2 hours and allowed to cool over 16 hours. Water was added (20 ml) and the DCM evaporated in vacuo to precipitate a white solid from the remaining aqueous solution. This was collected by filtration and dried in vacuo to afford the title compound as a white solid (314 mg, 0.77 mmol, 81%).

¹H-NMR (DMSO-d₆, 400 MHz) δ 1.16 (m, 1H), 1.44 (m, 2H), 1.59 (m, 1H), 1.68-1.90 (m, 4H), 2.57 (m obscured by DMSO peak, assumed 2H), 4.62 (d, 1H), 4.74 (d, 1H), 6.05 (d, 1H), 7.15 (s, NH), 7.19 (d, 1H), 7.32 (m, 1H), 7.40 (m, 1H), 7.43 (d, 1H), 8.21 (s, NH).

LCMS m/z (ESI) 409 [M+H]⁺

### Preparation 34

### {2-[(8′-chloro-2′-oxo-2′,3′-dihydro-1′H-spiro[cyclohexane-1,4′-quinazolin]-5′-yl)oxy]-3-fluorophenyl}acetonitrile

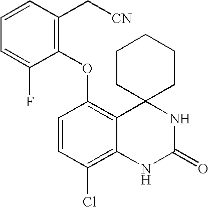

To a stirred solution of sodium cyanide (57 mg, 1.15 mmol) in DMSO (2 ml) was added the product of Preparation 33 (314 mg, 0.77 mmol) in DMSO (3 ml) dropwise. The resulting solution was stirred at room temperature for 18 hours. Water (15 ml) was added, resulting in a white precipitate that was collected by filtration and dried in vacuo to afford the title compound as a white solid (275 mg, 0.69 mmol, 89%).

¹H-NMR (DMSO-d₅, 400 MHz) δ 1.20 (m, 1H), 1.44 (m, 2H), 1.57 (m, 1H), 1.70-1.93 (m, 4H), 2.57 (m obscured by DMSO peak, assumed 2H), 3.98 (s, 2H), 6.05 (d, 1H), 7.11 (s, NH), 7.19 (d, 1H), 7.30-7.41 (m, 3H), 8.22 (s, NH).

LCMS m/z (ESI) 400 [M+H]⁺

### Preparation 35

### 8′-chloro-5′-(2-hydroxyphenoxy)-1′H-spiro[cyclohexane-1,4′-quinazolin]-2′(3′H)-one

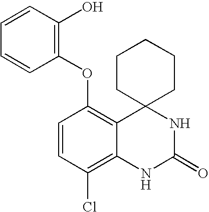

### a) 2-[(8′-chloro-2′-oxo-2′,3′-dihydro-1′H-spiro[cyclopentane-1,4′-quinazolin]-5′-yl)oxy]phenyl formate

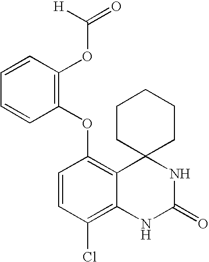

m-Chloroperbenzoic acid (136 mg, 0.1.54 mmol) was added in one portion to the product of Preparation 20 (190 mg, 0.512 mmol) suspended in DCM (3 ml) and the resulting solution stirred at room temperature under N₂ for 18 hours. Saturated Na₂SO₃ was added to destroy excess oxidant. The solution was basified by addition of NaHCO₃ and extracted with DCM three times. The organic layer was backwashed with brine, dried over MgSO₄ and concentrated in vacuo to give 185 mg impure title compound. Attempted purification with polymer supported carbonate unsuccessful; therefore formyl ester was recovered from the polymer by washing with HCl in ethanol to give the title compound (151 mg) which was used without further purification in step b).

### b) 8′-chloro-5′-(2-hydroxyphenoxy)-1′H-spiro[cyclohexane-1,4′-quinazolin]-2′(3′H)-one

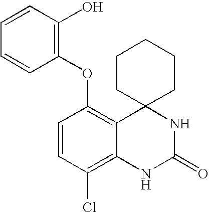

The product of step a) was taken up in methanol (3 ml) and sodium methoxide (50 mg) added and stirred for about 1 hour. The reaction was concentrated in vacuo and partitioned between DCM and 2NHCl. The organic phase was washed with brine, dried over MgSO₄ and evaporated in vacuo. Purification on a 4 g ISCO® cartridge eluting with a gradient of 100% heptane to 100% ethyl acetate afforded the still impure title compound (122 mg, 0.339 mmol, 66%) which was used directly in the next step without further treatment.

LRMS m/z (APCI) 357 [M−H]+, (ESI) 357 [M−H]⁻

### Preparation 36

### 8′-chloro-5′-(4-hydroxyphenoxy)-1′H-spiro[cyclohexane-1,4′-quinazolin]-2′(3′H)-one

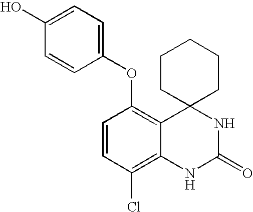

The title compound (57 mg, 0.16 mmol, 31%) was prepared in an analogous manner to Preparation 35 starting with the product of Preparation 21 (190 mg, 0.51 mmol).

¹H-NMR (CDCl₃, 400 MHz) δ 1.22-1.30 (m, 2H), 1.46-1.56 (m, 1H), 1.64-1.73 (m, 3H), 1.87-1.91 (m, 2H), 2.47-2.55 (m, 2H), 5.56 (s, 1H), 6.31 (d, 1H), 6.82-6.89 (m, 4H), 7.06 (broad s, 1H), 7.10 (d, 1H).

LRMS m/z (APCI) 359 [M+H]⁺.

### Preparation 37

### 2-[(8′-chloro-2′-oxo-2′,3′-dihydro-1′H-spiro[cyclohexane-1,4′-quinazolin]-5′-yl)oxy]-3-fluoro-N-hydroxybenzenecarboximidamide

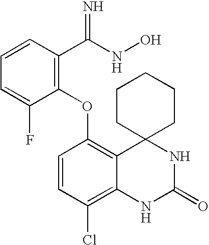

Potassium tert-butoxide (291 mg, 2.59 mmol) was added in portions to a suspension of hydroxylamine hydrochloride in DMSO (1.7 ml) at room temperature and stirred for 30 minutes. The product of Preparation 6 was added in one portion to give a turbid solution which was heated to 60° C. for 18 hours, cooled to room temperature, water added and the suspension agitated in a sonic bath. The product was collected by filtration and air dried to give the title compound as a white solid (100 mg, 0.238 mmol, 92%).

¹H-NMR (DMSO-d₆, 400 MHz) δ 1.19-1.27 (m, 1H), 1.46-1.84 (m, 7H), 2.55-2.63 (m, 2H), 5.75 (s, 2H), 6.05 (d, 1H), 7.07 (broad s, 1H), 7.15 (d, 1H), 7.34-7.46 (m, 3H), 8.13 (broad s, 1H), 9.46 (s, 1H).

LRMS m/z (APCI) 419 [M+H]⁺

1. A compound of formula (I): 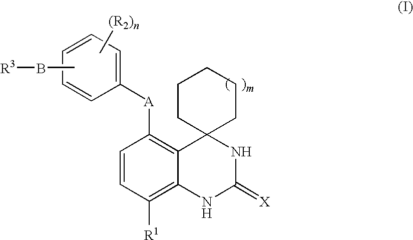
wherein:
m is 0, 1 or 2;
n is 0, 1, 2 or 3;
X is O, S or N—CN;
R¹ is halogen or CN;
A is a single bond, CH₂, O or S;
B is a single bond, CH₂ or OCH₂;
each R² is independently halogen, (C₁₋₆)alkyl (optionally substituted by 1 to 3 fluorine atoms), OH, (C₁₋₆)alkoxy, (C₁₋₆)alkylthio or CN;
R³ is selected from the following groups (i) to (x): 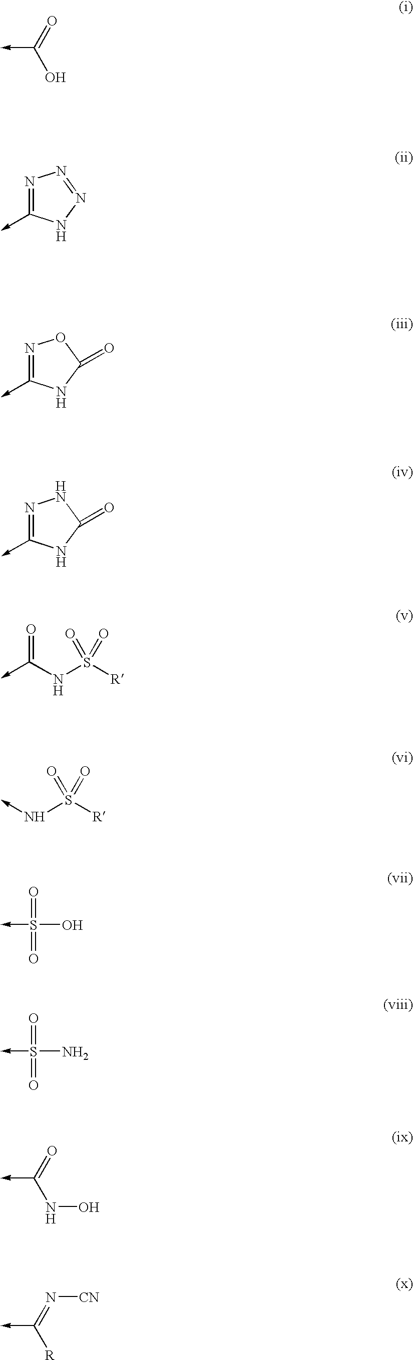
R is H or (C₁₋₆)alkyl (optionally substituted by 1 to 3 fluorine atoms);
R′ is (C₁₋₆)alkyl (optionally substituted by 1 to 3 fluorine atoms);
or a pharmaceutically acceptable salt, solvate, polymorph or prodrug thereof.

2. A compound, or a pharmaceutically acceptable salt, solvate, polymorph or prodrug thereof, according to claim 1, wherein m is 0 or 1.

3. A compound, or a pharmaceutically acceptable salt, solvate, polymorph or prodrug thereof, according to claim 1 or claim 2, wherein n is 0 or 1.

4. A compound, or a pharmaceutically acceptable salt, solvate, polymorph or prodrug thereof, according to any one of claims 1 to 3, wherein X is O.

5. A compound, or a pharmaceutically acceptable salt, solvate, polymorph or prodrug thereof, according to any one of claims 1 to 4, wherein R¹ is F or Cl.

6. A compound, or a pharmaceutically acceptable salt, solvate, polymorph or prodrug thereof, according to any one of claims 1 to 5, wherein A is a single bond or O.

7. A compound, or a pharmaceutically acceptable salt, solvate, polymorph or prodrug thereof, according to any one of claims 1 to 6, wherein B is a single bond.

8. A compound, or a pharmaceutically acceptable salt, solvate, polymorph or prodrug thereof, according to any one of claims 1 to 7, wherein R² is F or Cl.

9. A compound, or a pharmaceutically acceptable salt, solvate, polymorph or prodrug thereof, according to any one of claims 1 to 8, wherein R³ is a group (i), (ii), (iii), (iv), (v) or (vi).

10. A compound according to claim 1, selected from:
5-[(8′-chloro-2′-oxo-2′,3′-dihydro-1′H-spiro[cyclohexane-1,4′-quinazolin]-5′-yl)]-2-fluorobenzoic acid;
3-(8′-chloro-2-oxo-2′,3′-dihydro-1′H-spiro[cyclohexane-1,4′-quinazolin]-5′-ylbenzoic acid;
5-[(8′-chloro-2′-oxo-2′,3′-dihydro-1′H-spiro[cyclohexane-1,4′-quinazolin]-4′-yl)]-2-fluorobenzoic acid;
8′-chloro-5′-[4-fluoro-3-(2H-tetrazol-5-yl)phenyl]-1′H-spiro[cyclohexane-1,4′-quinazolin]-2′(3′H)-one;
[3-(8′-chloro-2′-oxo-2′,3′-dihydro-1′H-spiro[cyclohexane-1,4′-quinazolin]-5′-yl)phenoxy]acetic acid;
2-{(8′-chloro-2′-oxo-2′,3′-dihydro-1′H-spiro[cyclohexane-1,4′-quinazolin]-5′-yl)oxy}-3-fluorobenzoic acid;
2-{(8′-chloro-2′-oxo-2′,3′-dihydro-1′H-spiro[cyclopentane-1,4′-quinazolin]-5′-oxy}-3-fluorobenzoic acid;
3-chloro-2-{(8′-chloro-2′-oxo-2′,3′-dihydro-1′H-spiro[cyclohexane-1,4′-quinazolin]-5′-yl)oxy}benzoic acid;
3-chloro-2-{(8′-fluoro-2′-oxo-2′,3′-dihydro-1′H-spiro[cyclohexane-1,4′-quinazolin]-5′-yl)oxy}benzoic acid;
8′-chloro-5′-[2-fluoro-6-(2H-tetrazol-5-yl)phenoxy]-1′H-spiro[cyclohexane-1,4′-quinazolin]-2′(3′H)-one;
8′-chloro-5′-[4-fluoro-2-(1H-tetrazol-5-yl)phenoxy]-1′H-spiro[cyclohexane-1,4′-quinazolin]-2′(3′H)-one;
8′-chloro-5′-[6-fluoro-2-(1H-tetrazol-5-yl)phenoxy]-1′H-spiro[cyclohexane-1,4′-quinazolin]-2′(3′H)-one;
8′-chloro-5′-[4-fluoro-2-(1H-tetrazol-5-yl)phenoxy]-1′H-spiro[cyclopentane-1,4′-quinazolin]-2′(3′H)-one;
8′-chloro-5′-[6-fluoro-2-(1H-tetrazol-5-yl)phenoxy]-1′H-spiro[cyclopentane-1,4′-quinazolin]-2′(3′H)-one;
8′-chloro-5′-[6-chloro-2-(1H-tetrazol-5-yl)phenoxy]-1′H-spiro[cyclopentane-1,4′-quinazolin]-2′(3′H)-one;
8′-chloro-5′-[2-(1H-tetrazol-5-yl)phenoxy]-1′H-spiro[cyclopentane-1,4′-quinazolin]-2′(3′H)-one;
8′-chloro-5′-[2-(1H-tetrazol-5-yl)phenoxy]-1′H-spiro[cyclohexane-1,4′-quinazolin]-2′(3′H)-one;
8′-chloro-5′-[2-fluoro-6-(5-oxo-4,5-dihydro-1,2,4-oxadiazol-3-yl)phenoxy]-1′H-spiro[cyclohexane-1,4′-quinazolin]-2′(3′H)-one;
8′-chloro-5′-[2-fluoro-6-(5-oxo-4,5-dihydro-1H-1,2,4-triazol-3-yl)phenoxy]-1′H-spiro[cyclohexane-1,4′-quinazolin]-2′(3′H)-one;
2-[(8′-chloro-2′-oxo-2′,3′-dihydro-1′H-spiro[cyclohexane-1,4′-quinazolin]-5′-yl)oxy]-3-fluoro-N-(methylsulfonyl)benzamide;
N-{2-[(8′-chloro-2′-oxo-2′,3′-dihydro-1′H-spiro[cyclohexane-1,4′-quinazolin]-5′-yl)oxy]-3-fluorophenyl}-1,1,1-trifluoromethanesulfonamide;
{2-[(8′-chloro-2′-oxo-2′,3′-dihydro-1′H-spiro[cyclohexane-1,4′-quinazolin]-5′-yl)oxy]-3-fluorophenyl}acetic acid;
{2-[(8′-chloro-2′-oxo-2′,3′-dihydro-1′H-spiro[cyclohexane-1,4′-quinazolin]-5′-yl)oxy]phenoxy}acetic acid;
{4-[(8′-chloro-2′-oxo-2′,3′-dihydro-1′H-spiro[cyclohexane-1,4′-quinazolin]-5′-yl)oxy]phenoxy}acetic acid;
methyl 2-[(8′-chloro-2′-oxo-2′,3′-dihydro-1′H-spiro[cyclohexane-1,4′-quinazolin]-5′-yl)oxy]-3-fluorobenzoate;
or a pharmaceutically acceptable salt, solvate, polymorph or prodrug thereof.

11. A compound selected from:
8′-chloro-5′-[2-fluoro-6-(2H-tetrazol-5-yl)phenoxy]-1′H-spiro[cyclohexane-1,4′-quinazolin]-2′(3′H)-one;
8′-chloro-5′-[4-fluoro-2-(1H-tetrazol-5-yl)phenoxy]-1′H-spiro[cyclohexane-1,4′-quinazolin]-2′(3′H)-one;
8′-chloro-5′-[6-fluoro-2-(1H-tetrazol-5-yl)phenoxy]-1′H-spiro[cyclohexane-1,4′-quinazolin]-2′(3′H)-one;
8′-chloro-5′-[4-fluoro-2-(1H-tetrazol-5-yl)phenoxy]-1′H-spiro[cyclopentane-1,4′-quinazolin]-2′(3′H)-one;
8′-chloro-5′-[6-fluoro-2-(1H-tetrazol-5-yl)phenoxy]-1′H-spiro[cyclopentane-1,4′-quinazolin]-2′(3′H)-one;
8′-chloro-5′-[6-chloro-2-(1H-tetrazol-5-yl)phenoxy]-1′H-spiro[cyclopentane-1,4′-quinazolin]-2′(3′H)-one;
8′-chloro-5′-[2-(1H-tetrazol-5-yl)phenoxy]-1′H-spiro[cyclopentane-1,4′-quinazolin]-2′(3′H)-one;
8′-chloro-5′-[2-(1H-tetrazol-5-yl)phenoxy]-1′H-spiro[cyclohexane-1,4′-quinazolin]-2′(3′H)-one;
or a pharmaceutically acceptable salt, solvate, polymorph or prodrug thereof.

12. A pharmaceutical composition comprising a compound, or a pharmaceutically acceptable salt, solvate, polymorph or prodrug thereof, according to any one of claims 1 to 11, and a pharmaceutically acceptable carrier or diluent.

13. A compound, or a pharmaceutically acceptable salt, solvate, polymorph or prodrug thereof, according to any one of claims 1 to 11, for use as a medicament.

14. Use of a compound, or a pharmaceutically acceptable salt, solvate, polymorph or prodrug thereof, according to any one of claims 1 to 11, in the manufacture of a medicament for the treatment of diseases or conditions for which therapy by a PDE7 inhibitor is relevant.

15. Use according to claim 14, wherein the disease or condition is pain.

16. Use according to claim 15, wherein the pain is neuropathic pain.

17. A compound, or a pharmaceutically acceptable salt, solvate, polymorph or prodrug thereof, according to any one of claims 1 to 11, for the treatment of diseases or conditions for which therapy by a PDE7 inhibitor is relevant.

18. A compound, or a pharmaceutically acceptable salt, solvate, polymorph or prodrug thereof, according to claim 17, wherein the disease or condition is pain.

19. A compound, or a pharmaceutically acceptable salt, solvate, polymorph or prodrug thereof, according to claim 18, wherein the pain is neuropathic pain.

20. A method of treating a disease or condition for which therapy by a PDE7 inhibitor is relevant, comprising administering an effective amount of a compound, or a pharmaceutically acceptable salt, solvate, polymorph or prodrug thereof, according to any one of claims 1 to 11.

21. A method according to claim 20, wherein the disease or condition is pain.

22. A method according to claim 21, wherein the pain is neuropathic pain.
